(19) United States
(12) Patent Application Publication
(10) Pub. No.: US 2020/0063153 A1
(43) Pub. Date: Feb. 27, 2020

(54) METHODS AND COMPOSITIONS FOR IMPROVING FORAGE PRODUCTION OR QUALITY IN ALFALFA PLANTS

(71) Applicant: Noble Research Institute, LLC, Ardmore, OK (US)

(72) Inventors: Maofeng Chai, Ardmore, OK (US); Zengyu Wang, Ardmore, OK (US)

(21) Appl. No.: 16/439,548

(22) Filed: Jun. 12, 2019

Related U.S. Application Data
(60) Provisional application No. 62/684,121, filed on Jun. 12, 2018.

(51) Int. Cl. C12N 15/82

(52) U.S. Cl. CPC C12N 15/8261; C12N 15/8218

(57) ABSTRACT

The disclosure relates to the alfalfa FLOWERING LOCUS T (MsFT) gene. Provided by the disclosure are modified plants that express the MsFT gene. Also provided by the disclosure are methods and compositions for down-regulating the expression of the polypeptide encoded by the MsFT gene, including methods employing RNA interference and genome-editing. Still further provided by the disclosure are modified alfalfa plants that exhibit down-regulated expression of the polypeptide encoded by the MsFT gene, delayed flowering, improved vigor, and improved forage yield or quality.

### CROSS-REFERENCE TO RELATED APPLICATIONS

This application claims the benefit of priority of U.S. Provisional Application Ser. No. 62/684,121, filed Jun. 12, 2018, the entire disclosure of which is incorporated herein by reference.

### INCORPORATION OF SEQUENCE LISTING

The sequence listing that is contained in the file named NBLE096US_revised_ST25.txt, which is 9,934 bytes (measured in MS-Windows) and was created on Nov. 18, 2019, is filed herewith by electronic submission and incorporated herein by reference.

### FIELD OF THE INVENTION

The present invention relates to the field of biotechnology. More specifically, the invention relates to compositions and methods for producing alfalfa plants that exhibit improved forage production or quality.

### BACKGROUND

Alfalfa (Medicago sativa L.), also known as lucerne, is a valuable forage legume. Alfalfa is grown worldwide as forage for livestock, especially cattle, and is one of the most important forage crops worldwide and the fourth most valuable crop in the USA. Alfalfa is among the highest-yielding forage crop species. It is the combination of high yield and nutritional quality, however, that makes alfalfa such a valuable crop. As a perennial species with nitrogen fixation ability, alfalfa can be harvested multiple times for many years with little or no nitrogen fertilizer. Alfalfa is most often harvested as hay, but is also grazed, made into silage, and fed as greenchop. Alfalfa is primarily used to feed high-producing dairy cows, but is also a food source for beef cattle, horses, sheep, goats, rabbits, and poultry. Humans consume alfalfa sprouts and incorporate dehydrated alfalfa into dietary supplements.

Alfalfa breeding is particularly complicated because alfalfa has an autotetraploid genome and is frequently self-incompatible, i.e., an obligate outcrossing species. When alfalfa is selfed, little or no seed is produced. Typically, fewer than five percent of selfed crosses produce seed. If any seed is produced through self-pollination, it generally does not germinate, and the few seeds that germinate generally have reduced vigor or may stop growing. Producing true breeding alfalfa parents is therefore not possible by traditional means, and alfalfa breeders should be cautious when cross-breeding very small alfalfa populations, as inbreeding depression can occur, agronomic performance may decline, and traits of interest may be lost. Many cultivars have been generated by traditional breeding with very modest improvement in yield and disease resistance. However, partially due to the long breeding cycle and its extremely polymorphic populations, traditional alfalfa breeding programs have been much slower compared to annual crops.

### SUMMARY

One aspect of the present invention relates to a genetically engineered alfalfa plant comprising a modification in its genome that results in artificially down-regulated expression of the FLOWERING LOCUS T (FT) gene, wherein the modification confers to said plant improved forage production, quality, or, digestibility, or a combination thereof when compared to a second plant of the same variety lacking the modification. In certain embodiments, a genome modification may comprise a deletion, a substitution, or an insertion, or a combination thereof. In other embodiments, the genome modification may be made to any component of the genome including, for example, endogenous genes and regulatory elements thereof. In specific embodiments, the genome modification is to the MsFT gene. In other embodiments, the genome modification comprises a recombinant DNA molecule integrated into the genome of the plant that comprises all or a portion of a nucleic acid sequence at least 95% identical to SEQ ID NO:1, wherein the transcription of all or a portion of the recombinant DNA molecule suppresses the expression of the MsFT gene in the genetically engineered plant through an RNA interference pathway. In further embodiments, the genome modification may be produced by any method routine in the art, for example, irradiation, transposon insertion, chemical mutagenesis, genetic transformation, or genome-editing, or a combination thereof.

Another aspect of the present invention relates to a genetically engineered alfalfa plant comprising a modification in its genome that results in artificially down-regulated expression of the FLOWERING LOCUS T (FT) gene, wherein the modification confers to said plant improved forage production, quality, or digestibility, or a combination thereof when compared to a second plant of the same variety lacking the modification, and wherein the plant is an agronomically acceptable plant that exhibits increased vigor, biomass, percent dry matter, leaf to stem ratio, crude protein, or percent neutral detergent fiber digestibility, or decreased percent acid detergent fiber, percent acid detergent lignin, or percent neutral detergent fiber, or a combination thereof when compared to a second plant of the same variety lacking the modification.

Another aspect of the present invention relates to a recombinant DNA molecule comprising a promoter functional in alfalfa operably linked to all or a portion of a polynucleotide molecule at least 95% identical to SEQ ID NO:1 in an antisense or sense orientation or both, wherein the transcription of all or a portion of the recombinant DNA molecule in an alfalfa plant results in suppressing the expression of the MsFT gene in said plant. In certain embodiments of the invention, transcription of all or a portion of the recombinant DNA molecule may produce a double stranded RNA. In more specific embodiments, the recombinant DNA molecule may comprise a DNA sequence with at least 95% sequence identity to SEQ ID NOs:4 or 5, or a fragment thereof. In even more specific embodiments, all or a portion of the recombinant DNA molecule may correspond to the segment B region of the MsFT gene. In still further embodiments, the recombinant DNA molecule may be operably linked to a heterologous promoter. In other embodiments, the recombinant DNA molecule may comprise a vector useful in molecular cloning, such as a pENTR™/D-TOPO® vector, a transduction vector, a transfection vector, or a transformation vector, such as the pANDA35HK binary T-DNA vector, or a combination thereof.

Yet another aspect of the invention provides for a transgenic alfalfa plant, plant part, seed, or plant cell that comprises a recombinant DNA molecule comprising a promoter functional in alfalfa operably linked to all or a portion of a polynucleotide molecule at least 95% identical to SEQ ID NO:1 in an antisense or sense orientation or both, wherein the transcription of all or a portion of the recombinant DNA molecule in an alfalfa plant results in suppressing the expression of the MsFT gene in said plant. In specific embodiments, the plant exhibits improved forage production, quality, digestibility, or a combination thereof when compared to a plant of the same variety lacking the recombinant DNA molecule. In even more specific embodiments, the plant may exhibit increased vigor, biomass, percent dry matter, leaf to stem ratio, crude protein, or percent neutral detergent fiber digestibility, or decreased percent acid detergent fiber, percent acid detergent lignin, or percent neutral detergent fiber, or a combination thereof when compared to the plant of the same variety lacking the recombinant DNA molecule.

Still yet another aspect of the invention provides a method for producing a transgenic plant, wherein the method comprises the following steps: (a) transforming a plant cell with the recombinant DNA molecule comprising a promoter functional in alfalfa operably linked to all or a portion of a polynucleotide molecule at least 95% identical to SEQ ID NO:1 in an antisense or sense orientation or both, wherein the transcription of all or a portion of the recombinant DNA molecule in an alfalfa plant results in suppressing the expression of the MsFT gene in said plant; and (b) regenerating a transgenic plant from the transformed plant cell. In certain embodiments, such a method may further comprise the following steps: (a) transforming a plurality of plant cells with the recombinant DNA molecule; (b) regenerating a plurality of transgenic plants from the transformed plant cells; and (c) screening the plurality of transgenic plants to select at least a first plant comprising improved forage production, forage quality or forage digestibility. In certain embodiments, the invention provides for alfalfa plants produced by any of the foregoing methods contemplated by the invention.

In yet another embodiment, the invention provides a method of producing an alfalfa plant exhibiting improved forage production, quality, or digestibility, or a combination thereof, wherein the method comprises crossing a plant contemplated by the invention with itself or another alfalfa plant. In a further embodiment, the invention provides a method of generating a modified alfalfa plant exhibiting improved forage production, quality, or digestibility, or a combination thereof, wherein the method comprises introducing a mutation into the MsFT gene in the alfalfa plant that results in reduced expression of said gene. In certain embodiments, such a method comprises introducing said mutation using genome-editing or genetic transformation.

### BRIEF DESCRIPTION OF THE DRAWINGS

FIG. 1 shows alignment of the MtFT and MsFT coding sequences. The coding sequence of the M. truncatula FT (MtFT) gene (SEQ ID NO:10), top, and M. sativa FT (MsFT) gene (SEQ ID NO:2), middle, are aligned, and a consensus sequence (SEQ ID NO:11) of the conserved residues between the two coding sequences is provided, below. The coding sequences of the MtFT and MsFT gene have 96.43% sequence identity. The 248 bp sequence of the MsFT coding sequence (SEQ ID NO:4) that was used in engineering the pANDA35HK-MsFT-RNAi:FT242 binary transformation vector corresponds to nucleotides 254-501 of the MsFT coding sequence as it is enumerated here. Further, this 248 bp sequence encompasses the highly-conserved segment B region of the MsFT coding sequence that corresponds to nucleotides 382-423 of the MsFT coding sequence.

FIG. 2 shows a map of the T-DNA segment of the binary pANDA35HK-MsFT-RNAi:FT242 transformation vector. The map details the relative positioning and directionality of the Kanamycin resistance (NPT II), Hygromycin resistance (HPT), and MsFT RNAi expression cassettes with respect to the right border (RB) and left border (LB) of the T-DNA segment within the pANDA35HK-MsFT-RNAi:FT242 binary transformation vector. Functional components comprising the MsFT RNAi expression cassette are highlighted including the Cauliflower mosaic virus (35SCaMV) promoter (35S pro), a 248 bp segment of the MsFT coding sequence (MSFT248 bp) inserted in the antisense direction (SEQ ID NO:5), a GUS linker, the same 248 bp segment of the MsFT coding sequence inserted in the sense direction (SEQ ID NO:4), and a terminator (T).

FIG. 3 shows the relative MsFT expression level in 8 exemplary transgenic MsFT-knockdown alfalfa lines. Total RNA of the transgenic lines and corresponding wildtype controls was extracted from fully-expanded, mature leaves. For each line, 2.0 μg of the extracted RNA was reverse transcribed into cDNA. The cDNA was DNase treated and raw transcript levels were determined by RT-PCR. Relative transcript levels were calculated by normalizing the raw transcript levels against the raw transcript levels of UBQ. The relative expression level of MsFT in the 8 exemplary transgenic MsFT-knockdown alfalfa lines was decreased by more than 80% relative to that of the corresponding wildtype controls.

FIG. 4 shows the delayed flowering of 8 exemplary transgenic MsFT knockdown alfalfa lines. Plants from the 8 exemplary transgenic MsFT knockdown alfalfa lines were transitioned to soil and maintained under the following greenhouse conditions: 24° C. at day/22° C. at night, 16 h/8 h photoperiod, and relative humidity of 70%. Plants of the corresponding wildtype lines were also transplanted at this time. These plants were observed daily to determine the duration in days between transplanting and flowering. Plants of the 4 exemplary Regen SY4D-derived transgenic MsFT knockdown alfalfa lines exhibited a delay in flowering time that ranged from 16 to 22 days when compared against wildtype plants, and plants of the 4 exemplary R2336-derived transgenic MsFT knockdown alfalfa lines exhibited a delay in flowering time that was typically 22 days when compared against wildtype plants.

FIG. 5 shows the fresh and dry biomass yield and leaf to stem ratio of 8 exemplary transgenic MsFT knockdown alfalfa lines. FIG. 5A shows the fresh weight (g) of biomass harvested from plants of the 4 exemplary Regen SY4D-derived transgenic MsFT knockdown alfalfa lines and wildtype Regen SY4D plants. FIG. 5B shows dry weight (g) of biomass harvested from plants of the 4 exemplary Regen SY4D-derived transgenic MsFT knockdown alfalfa lines and wildtype Regen SY4D plants. FIG. 5C shows the leaf to stem ratio of the biomass harvested from plants of the 4 exemplary Regen SY4D-derived transgenic MsFT knockdown alfalfa lines and wildtype Regen SY4D plants. FIG. 5D shows fresh weight (g) of biomass harvested from plants of the 4 exemplary R2336-derived transgenic MsFT knockdown alfalfa lines and wildtype R2336 plants. FIG. 5E shows dry weight (g) of biomass harvested from plants of the 4 exemplary R2336-derived transgenic MsFT knockdown alfalfa lines and wildtype R2336 plants. FIG. 6F shows the leaf to stem ratio of the biomass harvested from plants of the 4 exemplary R2336-derived transgenic MsFT knockdown alfalfa lines and wildtype R2336 plants.

FIG. 6 shows estimated forage quality of 8 exemplary transgenic MsFT knockdown alfalfa lines. FIG. 6A shows the estimated crude protein percentage of the biomass harvested from plants of the 4 exemplary Regen SY4D-derived transgenic MsFT knockdown alfalfa lines and wildtype Regen SY4D plants. FIG. 6B shows the estimated acid detergent fiber percentage of the biomass harvested from plants of the 4 exemplary Regen SY4D-derived transgenic MsFT knockdown alfalfa lines and wildtype Regen SY4D plants. FIG. 6C shows the estimated acid detergent lignin percentage of the biomass harvested from plants of the 4 exemplary Regen SY4D-derived transgenic MsFT knockdown alfalfa lines and wildtype Regen SY4D plants. FIG. 6D shows the estimated crude protein percentage of the biomass harvested from plants of the 4 exemplary R2336-derived transgenic MsFT knockdown alfalfa lines and wildtype R2336 plants. FIG. 6E shows the estimated acid detergent fiber percentage of the biomass harvested from plants of the 4 exemplary R2336-derived transgenic MsFT knockdown alfalfa lines and wildtype R2336 plants. FIG. 6F shows the estimated acid detergent lignin percentage of the biomass harvested from plants of the 4 exemplary R2336-derived transgenic MsFT knockdown alfalfa lines and wildtype R2336 plants.

### BRIEF DESCRIPTION OF THE SEQUENCES

SEQ ID NO:1 Endogenous MsFT gene.

SEQ ID NO:2 Sense MsFT cDNA sequence.

SEQ ID NO:3 Antisense MsFT cDNA sequence.

SEQ ID NO:4 Sense MsFT-RNAi insert.

SEQ ID NO:5 Antisense MsFT-RNAi insert.

SEQ ID NO:6 MsFT mRNA sequence.

SEQ ID NO:7 MsFT sequence.

SEQ ID NO:8 Forward primer used to amplify MsFT-RNAi insert.

SEQ ID NO:9 Reverse primer used to amplify MsFT-RNAi insert.

SEQ ID NO:10 MtFT cDNA sequence.

SEQ ID NO:11 Consensus between MtFT cDNA sequence (SEQ ID NO:10) and Sense MsFT cDNA sequence (SEQ ID NO:2).

### DETAILED DESCRIPTION OF THE INVENTION

In specific embodiments, the invention represents a significant advance in the art by providing alfalfa plant with suppressed FLOWERING LOCUS T (FT) gene expression that confers a surprising improvement in forage yield and quality. Plants prepared in accordance with the invention were shown to exhibit substantial decreases in acid detergent fiber (ADF) and acid detergent lignin (ADL) content when compared to corresponding wildtype controls. This indicates that the lines with suppressed FT gene expression exhibit improved digestibility, an important metric of alfalfa forage quality. In line with ADF and ADL metrics, neutral detergent fiber percent (NDF) was also decreased in transgenic alfalfa FT knockdown lines in comparison to controls. NDF represents the total amount of fiber in a forage, and decreased NDF like that achieved indicates the lines produce a forage that is more digestible as well as more consumable. Transgenic FT knockdown lines also exhibited increased neutral detergent fiber digestibility percent (NDFD), reflecting the rate at which the fiber fraction of the forage will be digested in the rumen. The fiber component of the forage achieved by the invention is therefore also more digestible. Moreover, the FT knockdown lines exhibited increased crude protein (CP) when compared to controls, which is indicative of improved nutritional quality.

The present disclosure therefore provides methods and compositions that surprisingly allow for improved alfalfa forage production. In particular embodiments disclosed herein, isolated recombinant nucleic acids comprising all or a part of the sequence of the M. sativa FT (MsFT) gene are provided, including the coding and/or any regulatory portions. Another embodiment provided herein is a recombinant DNA molecule that encodes a polynucleotide molecule, wherein the polynucleotide molecule, or a fragment thereof, is capable of hybridizing to an RNA molecule encoded by the MsFT gene and that is targeted to the RNA molecule through an RNA interference pathway. Another embodiment contemplated by the disclosure provides an alfalfa plant exhibiting artificially down-regulated expression of the polypeptide encoded by the MsFT gene and exhibits an improved trait selected from delayed flowering, improved vigor or improved forage production, quality, or digestibility, or a combination thereof. Still other embodiments of the invention comprise alfalfa plants in which the endogenous FT gene has been mutated to reduce the expression of the FT gene relative to a wild-type plant to yield an improvement in forage production or quality. In specific embodiments such plants can be generated by techniques such as gene-editing using the CRISP/Cas-9 or other systems for gene-editing. Also provided are methods of producing such alfalfa plants, as well as the plants produced thereby.

The finding that forage yield can be achieved by suppression of the FT gene is particularly surprising in that ablating the FT gene in Medicago truncatula FT (MtFT), a plant with a highly conserved genome structure and function with alfalfa, significantly reduces plant growth and biomass yield. The M. truncatula plants were shorter due to dramatically reduced elongation of main stem internodes and showed a more prostrate growth habit. In contrast, alfalfa plant engineered to suppress the MsFT gene exhibited unexpectedly improved yields and increased vigor. A further aspect of the invention therefore provides an alfalfa plant with suppressed FT gene expression, wherein the plant lacks a reduction in plant growth or biomass, or in still further aspects, is defined as exhibiting increased forage yield relative to a plant lacking the suppressed FT gene expression.

The majority of the alfalfa grown in the U.S.A. is used for hay production. A significant hurdle when harvesting alfalfa as forage is staging the harvest to optimize both yield and nutrient content. The nutrient value of alfalfa peaks prior to flowering and decreases as the plant matures further; however, alfalfa forage yield peaks when the plants are fully flowering. Therefore, small adjustments in harvest timing can significantly impact the forage quality at the detriment of forage yield and vice versa. Alfalfa is one of the most important forage crops worldwide and the fourth most valuable crop in the U.S.A. Technologies that mitigate or eliminate this tradeoff between the yield and quality of an alfalfa forage at the time of harvest are therefore of significant economic importance to farmers that produce alfalfa commodity products and the downstream industries that rely on those products. The plants and methods described herein reduce the previous need to coordinate alfalfa harvest timing so that the quality of the forage is not sacrificed to obtain greater yield.

In particular aspects, the invention therefore provides alfalfa plants with one or more improved characteristic selected from delayed flowering, improved vigor, and improved forage production, quality, digestibility, or a combination thereof. In specific embodiments, such an improved phenotype can be achieved using DNA-directed RNA interference. The current disclosure therefore provides recombinant DNA molecules capable of suppressing the alfalfa FT gene through an RNA interference-mediated pathway. In particular embodiments, such DNA molecules may comprise a sequence with at least 85%, 86%, 87%, 88%, 89%, 90%, 91%, 92%, 93%, 94%, 95%, 96%, 97%, 98%, 99%, or 100% sequence identity to SEQ ID NOs:1, 2, 3, 4, or 5, or a fragment thereof. The disclosure also provides recombinant DNA molecules, wherein the polynucleotide molecules encoded by these recombinant DNA molecules or complements thereof are defined as capable of hybridizing to an RNA molecule encoded by any of the coding sequences in the Sequence Listing. The disclosure further provides any DNA molecules that are complementary to the foregoing molecules as well as fragments thereof.

The disclosure further contemplates operably linking a recombinant DNA molecule provided herein to a heterologous promoter or other regulatory element. An exemplary promoter is a recognition site on a DNA sequence or group of DNA sequences that provides an expression control element for a structural gene or ncRNA and to which RNA polymerase specifically binds and initiates RNA synthesis (transcription) of that gene. In accordance with the disclosure, the heterologous promoter may be, but is not limited to, a constitutive promoter, an inducible promotor, a tissue-specific promoter, a cell-specific promoter, an organelle-specific promoter, or a developmentally regulated promoter. Also in accordance with the disclosure, a DNA sequence that serves a cell biology function may be, but is not limited to, a sequence encoding a nuclear localization sequence, an internal ribosome entry site, a sequence encoding an amino acid motif, e.g., a polyhistidine tag, and a sequence encoding a fluorescent protein. The disclosure also contemplates the foregoing DNA molecules as a component of a larger DNA molecule. For example, DNA molecules of the disclosure may be integrated into any type of plasmid, viral vector, or artificial chromosome, including, but not limited to, those used for cloning, expression, transfection, viral transduction, or transformation. In particular embodiments, plasmids, viral vectors, or artificial chromosomes contemplated by the disclosure may be specifically designed for transformation into a host cell, capable of replication in a host cell, or a combination thereof. The DNA molecules of the disclosure, when integrated into a larger DNA molecule, may also be operably linked to a heterologous promoter or a DNA sequence that serves a cell biology function, as described above, or integrated into the larger DNA molecule in such a manner as to operably link them to functional components contained within the larger DNA molecule, such as, a promoter, a DNA sequence that serves a cell biology function, or any other cis-acting regulatory element. In a specific embodiment, such a DNA construct contemplated by the disclosure may comprise a vector useful in molecular cloning, such as a pENTR™/D-TOPO® vector, a transduction vector, a transfection vector, or a transformation vector, such as the pANDA35HK binary T-DNA vector, or a combination thereof.

The disclosure further provides methods for generating transgenic plants by transformation with any of the DNA molecules provided by the disclosure. Many plant transformation methods are known in the art and may be used in accordance with the disclosure, for example, Agrobacterium-mediated and microprojectile bombardment transformation may be employed. In accordance with the disclosure, specific embodiments of these methods can further comprise a DNA detection step performed to detect a DNA molecule of the disclosure in the cells, protoplasts, regenerated plants, and the like that have been transformed with a DNA molecule of the disclosure, or a selection step that is performed to select cells, protoplasts, regenerated plants, and the like that have been transformed with a DNA molecule of the disclosure, or a combination thereof. Techniques for carrying out the selection and DNA detection steps described above are well known in the art and are compatible with the disclosure.

The disclosure also provides alfalfa plants engineered to exhibit artificially down-regulated FT gene expression and improved forage production, quality, or digestibility, or a combination thereof, when compared to a second alfalfa plant of the same variety that has not been engineered according to the invention. In particular embodiments, alfalfa plants of the disclosure also exhibit delayed flowering. Metrics that demonstrate delayed flowering and improved forage production, quality, and digestibility are described herein and can be measured according to methods known in the art. In accordance with the disclosure, improved forage quality may be indicated by, for example, forage with an increased percentage of dry matter or crude protein, or a combination thereof; forage with a decreased percentage of acid detergent fiber, neutral detergent fiber, or acid detergent lignin, or a combination thereof; or any combination of the foregoing. In accordance with the disclosure, improved forage digestibility may be indicated by, for example, forage with an increased percentage of crude protein or neutral detergent fiber digestibility, or a combination thereof; or forage with a decreased percentage of acid detergent fiber, neutral detergent fiber, or acid detergent lignin, or any combination of the foregoing. In accordance with the disclosure, improved forage production may be indicated by, for example, an increased amount of the biomass to be harvested from an alfalfa plant, increased vigor, dry weight, fresh weight, or leaf to stem ratio, or a combination thereof. Further, improved forage production as contemplated by the invention may reflect improved growth phenotypes in alfalfa plants engineered to exhibit artificially down-regulated FT gene. In specific embodiments, improved growth phenotypes may be characterized, for example, by an increase in branching, an increase in leaf production or size, an increase in the rate of vegetative growth, or an elevated growth rate observed in one or more developmental stages. In accordance with the disclosure, growth phenotypes may be measured over any duration of time and during any number of developmental stages.

In accordance with the disclosure, down-regulated expression of a polypeptide encoded by the FT gene is decreased by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% relative to a control plant. In certain embodiments, the expression of the mRNA that is translated to produce the polypeptide encoded by the FT gene may be defined as down-regulated by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% relative to a control plant. The disclosure also contemplates the ranges between each percentage listed above. In certain embodiments, the down regulated expression may be due to “gene silencing” or “RNA silencing,” which as used herein indicates that the down-regulated expression of the polypeptide encoded by FT gene is a result of RNA interference.

The disclosure contemplates plants that may be decreased or increased in an agronomic characteristics described herein. A decrease or increase in an agronomic characteristic, as used herein, is a relative comparison between a plant disclosed herein and a plant that is the appropriate scientific negative control. The disclosure contemplates relative decreases and increases of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and 100% in any of these agronomics characteristics. The disclosure further provides for relative increases of 105%, 110%, 115%, 120%, 125%, 130%, 135%, 140%, 145%, 150%, 155%, 160%, 165%, 170%, 175%, 180%, 185%, 190%, 195%, 200%, 205%, 210%, 215%, 220%, 225%, 230%, 235%, 240%, 245%, 250%, 255%, 260%, 265%, 270%, 275%, 280%, 285%, 290%, 295%, 300%, 305%, 310%, 315%, 320%, 325%, 330%, 335%, 340%, 345%, 350%, 355%, 360%, 365%, 370%, 375%, 380%, 385%, 390%, 395%, 400%, 405%, 410%, 415%, 420%, 425%, 430%, 435%, 440%, 445%, 450%, 455%, 460%, 465%, 470%, 475%, 480%, 485%, 490%, 495%, and 500% in any of these agronomic characteristics. The evaluation of a specific agronomic characteristic contemplated by the disclosure is distinct from that of any other agronomic characteristic. The above contemplated relative increases and decreases may therefore be applied distinctly to any agronomic trait described herein, i.e., the disclosure contemplates that the above percentages may be applied to each agronomic trait separately and independently. The disclosure also contemplates the ranges between each percentage listed above.

In a specific embodiment of the disclosure, the alfalfa plants described above may comprise a genetic modification that enables artificially down-regulated expression of a polypeptide encoded by SEQ ID NO:1 and improved forage production, quality, digestibility, or a combination thereof when compared to a second plant of the same variety that does not comprise the genetic modification. In accordance with the disclosure, a genetic modification may be, but is not limited to, any DNA sequence difference, epigenetic difference, or combination thereof between two genomes of the same species in which one genome is identified as the modified genome and the other is identified as the unmodified genome and the DNA sequence or epigenetic difference is the result of applying genome modifying techniques to the unmodified genome to yield the modified genome. A genetic modification, as used herein, encompasses any insertion, deletion, or substitution of a nucleotide sequence of any size and nucleotide content, any epigenetic modification to any number of nucleotides, or a combination thereof. Also in accordance with the disclosure, a genetic modification may be made anywhere in the genome of an alfalfa plant; may be made specifically in or proximal to an endogenous gene or an ncRNA locus, or a regulatory element thereof or, even more specifically, may be made in or proximal to the endogenous MsFT gene or a regulatory element thereof. A genetic modification, as used herein, may also encompass introduction of one or more exogenous coding nucleic acids that do not integrate into the unmodified genome, yet are capable of autonomous replication. In specific embodiments, a genetic modification may comprise an indel that may result in a frameshift mutation, a missense mutation, a nonsense mutation, a neutral mutation, or a silent mutation.

The disclosure also contemplates genetic modifications that may function to, for example, alter or eliminate a function of a cis-regulatory element, provide exogenous control of an endogenous gene or ncRNA, repress or activate endogenous gene or ncRNA expression, or disrupt a post-transcriptional or post-translational processing of an RNA, for example, ncRNA, pre-mRNA, mRNA, or a polypeptide, respectively. In accordance with the disclosure, post-transcriptional RNA processes include, but are not limited to, alternative splicing, editing, polyadenylation, export and localization, and translation. In accordance with the disclosure, post-translational processes include, but are not limited to, post-translational modification, protein sorting, and proteasomal degradation.

In particular embodiments, the genetically modified alfalfa plants provided by the disclosure may be transgenic and therefore comprise transgenes. In preferred embodiments, these transgenes comprise recombinant DNA molecules, wherein the polynucleotide molecules encoded by the recombinant DNA molecules or fragments thereof, or complements of the foregoing, are capable of hybridizing to an RNA molecule encoded by SEQ ID NO:1 and are targeted to that RNA molecule through an RNA interference pathway. In a particular embodiment, these transgenes comprise a recombinant DNA molecule with a sequence with at least 85%, 86%, 87%, 88%, 89%, 90%, 91%, 92%, 93%, 94%, 95%, 96%, 97%, 98%, 99%, or 100% sequence identity to SEQ ID NOs:2, 3, 4, or 5. In other embodiments, the genetically modified plants of the provided in the disclosure may comprise a modification capable of activating expression or relieving suppression of an endogenous ncRNA that is effective at silencing expression of a polypeptide encoded by SEQ ID NO:1 through an RNAi pathway. The endogenous ncRNAs that are compatible with the embodiments provided in the disclosure may be, but are not limited to, miRNAs, piRNAs, or endo-siRNAs.

Yet another embodiment contemplated by the disclosure is a method for producing alfalfa plants that comprise a modification that enables artificially down-regulated expression of the FT gene and improved forage production, quality, digestibility, or a combination thereof when compared to a second plant of the same variety that does not comprise the genetic modification. In one embodiment, this method comprises a step in which a genetic modification is introduced into the genome of an alfalfa cell and a modified plant is regenerated therefrom. In particular embodiments, a plant is selected from a plurality of plants that comprises one or more beneficial phenotypic trait as described herein.

In particular embodiments, generating a modified cell may employ genome-editing or genetic transformation techniques to introduce a genetic modification into the genome of an alfalfa cell. Genome-editing techniques are well known in the art and contemplated for use herein. In accordance with the disclosure, genome-editing techniques the may be employed include, but are not limited to, TALENs, ZFNs, and the CRISPR/Cas and other systems. In specific embodiments that employ genome-editing techniques, the method may further comprise techniques that specifically introduce single-strand DNA breaks or double-strand DNA breaks. Other techniques for modifying the genome of any plant, including alfalfa plants, are well known in the art and are compatible with the embodiments of the disclosure. Additional techniques for genetically modifying a plant that may be used in specific embodiments are, for example, irradiation, transposon insertion, and chemical mutagenesis. Regeneration techniques are also well known in the art and compatible with the embodiments of the disclosure. Further, metrics of improved forage production, quality, and digestibility are described herein and compatible with the embodiments of the disclosure, as set forth.

The disclosure also provides methods for producing alfalfa plants that comprise a modification that enables artificially down-regulated expression of the FT gene and improved forage production, quality, digestibility, or a combination thereof when compared to a second plant of the same variety that does not comprise the genetic modification. The methods can comprise the steps of obtaining an alfalfa plant contemplated by the disclosure comprising a genetic modification, crossing that plant with itself or another plant to produce progeny plants, and selecting the progeny plants that comprise the genetic modification that enables artificially down-regulated expression of the FT gene and improved forage production, quality, digestibility, or a combination thereof. Methods of crossing alfalfa plants are well known in the art and described herein.

Further embodiments of contemplated by the disclosure provide cells, plants, and plant parts generated according to the methods described herein. In accordance with the disclosure, a plant part may be, but is not limited to, a protoplast, cell, meristem, root, pistil, anther, flower, seed, embryo, stalk, or petiole. In specific embodiments, a plant part may be any part of the plant from which another plant may arise. Nonregenerable plants parts are also provided herein. The disclosure further provides for progeny plants of any generation derived from the plants of the disclosure. Additionally, the disclosure contemplates methods of producing commodity products from the plants of the provided by the disclosure as well as the plants produced by the methods of provided by the disclosure. A commodity product may be, but is not limited to, protein concentrate, protein isolate, grain, starch, seeds, meal, flour, biomass, sprouts, forage, hay, greenchop, silage, and seed oil. The commodity products produced by these methods are also contemplated by the disclosure. In specific embodiments, the commodity products of the disclosure comprise a DNA molecule or genome modification described herein.

Embodiments discussed in the context of methods and/or compositions of the disclosure may be employed with respect to any other method or composition described herein. Thus, an embodiment pertaining to one method or composition may be applied to other methods and compositions of the disclosure as well.

### I. Genome Editing

Certain aspects of the disclosure relate to methods of modifying the genome of a plant using genome editing techniques. As used herein, “genome editing” and “genome-engineering” are terms used interchangeably and refer to the modification of a genome through mutagenesis. In specific embodiments, genome editing, as used herein, refers to modifying a genome with techniques that employ targeted mutagenesis to activate DNA repair pathways. These techniques include, but are not limited to, those that utilize endonucleases to generate single-strand and double-strand DNA breaks that activate DNA repair pathways. Genome editing techniques may also comprise systems that enable targeted editing at any genomic locus. These targeting systems include, but are not limited to, polypeptides, such as, Transcription Activator-Like Effectors (TALEs) and zinc fingers (ZFs), or nucleic acids, such as, Clustered Regularly Interspaced Short Palindromic Repeats/Cas (CRISPR/CAS) single guide RNAs or NgAgo (Argonaute) single strand DNAs.

For example, in plant genome engineering, endonucleases may be used to generate double-strand DNA breaks (DSBs) and activate genome repair pathways. These DSB repair pathways may repair the break cleanly, i.e., without altering the starting sequence, or, alternatively, induce a mutation through an error in repair. In some embodiments, genome editing is used to insert, delete, or substitute one or more base pairs at one or any combination of genetic loci. In some embodiments, a genome editing technique is used to create a mutation, for example, a point mutation or single nucleotide polymorphism.

In some embodiments the DSB repair pathway is non-homologous end-joining (NHEJ) or microhomology mediated end joining (MMEJ). During NHEJ, any nucleotide overhangs on the break ends are either resected or filled to form blunt ends that are ligated. During MMEJ, the break ends are processed to reveal overhangs comprising microhomology sequences that are then ligated together. The insertions or deletions resulting from the terminal end processing in both the NHEJ and MMEJ pathways can be referred to as indels. In some embodiments, the NHEJ or MHEJ that occurs can be relied upon to introduce a genome modification including, but not limited to, a silent mutation, a neutral mutation, a missense mutation, a nonsense mutations, or a frameshift mutation.

In other embodiments, the DSB repair pathway is homologous recombination (HR). During HR, a DSB is repaired using a template with sequences with homology to the DNA flanking the break, i.e., a homologous chromosome. In plant genome editing, a linear DNA polynucleotide flanked by sequences (e.g., of 50 base pairs or more) homologous to those flanking a targeted genomic locus, may be introduced into the genome when a DSB is repaired by HR. In some embodiments, this approach is used to introduce, substitute, or delete a DNA sequence at a genomic locus. Any DNA sequence of interest may be introduced, deleted, or substituted. An introduced or substituted DNA sequence may encode an RNA molecule with a specific activity or function, a DNA molecule with a specific activity or function (e.g., encoding a polypeptide, representing a detectable marker, etc.), a DNA molecule comprising cis-regulatory elements, or a DNA molecule encoding a polypeptide, a motif thereof, or domain thereof. In some embodiments, the nucleic acid encoding the linear DNA sequence that will act as the HR template is encoded by an expression vector. In some embodiments, the nucleic acid encoding the linear DNA sequence of interest is encoded by a DNA sequence separate from the expression vector. For example, and without limitation, the nucleic acid encoding a DNA sequence of interest may be a linear DNA polynucleotide that is co-transformed with an expression vector.

In some embodiments, single-strand breaks or “nicks” are introduced into the target DNA sequence. As used herein, the term “singe-strand break inducing agent” or “nickase” refers to any agent that can induce a single-strand break (SSB) in a DNA molecule. In some embodiments two SSBs are introduced into the target DNA to generate a DSB. These breaks may also be repaired by HR, NHEJ, or MMEJ. In some embodiments, sequence modifications occur at or near the SSB sites, which can include deletions or insertions that result in modification of the nucleic acid sequence, or integration of exogenous nucleic acids by HR or NHEJ.

In one aspect, a “modification” comprises the insertion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In another aspect, a “modification” comprises the deletion of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In a further aspect, a “modification” comprises the inversion of at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In still another aspect, a “modification” comprises the substitution of at least 1, at least 2, at least 3, at least 4, at least 5, at least 6, at least 7, at least 8, at least 9, at least 10, at least 15, at least 25, at least 50, at least 100, at least 200, at least 300, at least 400, at least 500, at least 750, at least 1000, at least 1500, at least 2000, at least 3000, at least 4000, at least 5000, or at least 10,000 nucleotides. In some embodiments, a “modification” comprises the substitution of an “A” for a “C”, “G” or “T” in a nucleic acid sequence. In some embodiments, a “modification” comprises the substitution of a “C” for an “A”, “G” or “T” in a nucleic acid sequence. In some embodiments, a “modification” comprises the substitution of a “G” for an “A”, “C” or “T” in a nucleic acid sequence. In some embodiments, a “modification” comprises the substitution of a “T” for an “A”, “C” or “G” in a nucleic acid sequence. In some embodiments, a “modification” comprises the substitution of a “C” for a “U” in a nucleic acid sequence. In some embodiments, a “modification” comprises the substitution of a “G” for an “A” in a nucleic acid sequence. In some embodiments, a “modification” comprises the substitution of an “A” for a “G” in a nucleic acid sequence. In some embodiments, a “modification” comprises the substitution of a “T” for a “C” in a nucleic acid sequence.

In some embodiments genome editing of a plant as described herein may encompass techniques that employ methods of targeting endonucleases to one or more genetic loci. In some embodiments, synthetic polypeptides, for example, Transcription Activator-Like Effectors (TALEs) and zinc fingers (ZFs), or nucleic acids, for example, Clustered Regularly Interspaced Short Palindromic Repeats/Cas (CRISPR/CAS) single guide RNAs or NgAgo (Argonaute) single strand DNAs, are used to target endonucleases to any genomic locus. The targeted endonucleases may catalyze a DSB at a target locus. Upon detecting these breaks, a cell may initiate any DSB repair pathway. In some embodiments, genome editing is carried out at more than one genomic locus simultaneously (i.e., multiplex genome engineering). In some embodiments, multiplex genome engineering may be used to remove a sequence of any size from the genome. In some embodiments, any combination and number of endonuclease targeting techniques may be used to target one or more genetic loci.

### A. RNA- and DNA-Guided Genome Editing Systems

In some embodiments, genome engineering of a plant as described herein may employ RNA-guided endonucleases including, but not limited to CRISPR/Cas systems. CRISPR/Cas systems have been described in U.S. Publication Nos. 2017/0191082 and 2017/0106025, which are incorporated herein by reference in their entirety. In some embodiments, a targeted genome modification as described herein comprises the use of at least one, at least two, at least three, at least four, at least five, at least six, at least seven, at least eight, at least nine, or at least ten RNA-guided nucleases. In some embodiments, a CRISPR/Cas9 system, a CRISPR/Cpf1 system, a CRISPR/CasX system, or a CRISPR/CasY system are alternatives that may be used to generate modifications to target sequences as described herein.

The CRISPR systems are based on RNA-guided endonucleases that use complementary base pairing to recognize DNA sequences at target sites. CRISPR/Cas systems are part of the adaptive immune system of bacteria and archaea, protecting them against invading DNA, such as viral DNA, by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA.

A prerequisite for cleavage is the presence of a conserved protospacer-adjacent motif (PAM) downstream of the target DNA, which usually has the sequence 5′-NGG-3′ but less frequently NAG. Specificity is provided by the so-called “seed sequence” approximately 12 bases upstream of the PAM, which should match between the RNA and target DNA. Cpf1 acts in a similar manner to Cas9, but Cpf1 does not require a tracrRNA. Specificity of the CRISPR/Cas system is based on an RNA-guide that use complementary base pairing to recognize target DNA sequences. In some embodiments, the site-specific genome modification enzyme is a CRISPR/Cas system. In an aspect, a site-specific genome modification enzyme provided herein can comprise any RNA-guided Cas endonuclease (non-limiting examples of RNA-guided nucleases include Cas1, Cas1B, Cas2, Cas3, Cas4, Cas5, Cash, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, or modified versions thereof); and, optionally, the guide RNA necessary for targeting the respective nucleases.

In some embodiments, an RNA-guided endonuclease is the DNA cleavage domain of a restriction enzyme fused to a deactivated Cas9 (dCas9), for example dCas9-Fok1. As used herein, a “dCas9” refers to an endonuclease protein with one or more amino acid mutations that result in a Cas9 protein without endonuclease activity, but retaining RNA-guided site-specific DNA binding. As used herein, a “dCas9-restriction enzyme fusion protein” is a dCas9 with a protein fused to the dCas9 in such a manner that the restriction enzyme is catalytically active on the DNA.

In some embodiments, genome editing of a plant as described herein may employ DNA-guided endonucleases including, but not limited to, NgAgo systems.

In one aspect, a method and/or composition provided herein comprises one or more, two or more, three or more, four or more, or five or more guide RNAs or DNAs. In another aspect, a CRISPR/CAS system, dCas9-restriction enzyme fusion protein, NgAgo system provided herein is capable of generating a targeted DSB in a target sequence as described herein. In one aspect, vectors comprising nucleic acids encoding one or more, two or more, three or more, four or more, or five or more guide RNAs or DNAs and the corresponding CRISPR/CAS system, dCas9-restriction enzyme fusion protein, NgAgo system are provided to a cell by transformation methods known in the art (e.g., without being limiting, viral transfection, particle bombardment, PEG-mediated protoplast transfection or Agrobacterium-mediated transformation).

### B. Transcription Activator-Like Effector Nucleases

In some embodiments, genome editing of a plant as described herein may employ Transcription Activator-Like Effector Nucleases (TALENs). TALENs have been described in U.S. Publication Nos. 2016/0369301 and 2015/0203871, which are incorporated herein by reference in their entirety, and are well known in the art. TALENs are artificial restriction enzymes generated by fusing the transcription activator-like effector (TALE) DNA binding domain to an endonuclease domain. In one aspect, the nuclease is selected from a group consisting of PvuII, MutH, TevI and FokI, AlwI, MlyI, SbfI, SdaI, StsI, CleDORF, Clo051, Pept071. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

TALEs can be engineered to bind practically any DNA sequence, such as a target sequence as described herein. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

In one aspect, a method and/or composition provided herein comprises one or more, two or more, three or more, four or more, or five or more TALENs. In another aspect, a TALEN provided herein is capable of generating a targeted DSB in a target sequence as described herein. In one aspect, vectors comprising polynucleotides encoding one or more, two or more, three or more, four or more, or five or more TALENs are provided to a cell by transformation methods known in the art (e.g., without being limiting, viral transfection, particle bombardment, PEG-mediated protoplast transfection or Agrobacterium-mediated transformation).

### C. Zinc Finger Nucleases

In some embodiments, genome engineering of a plant as described herein may employ Zinc Finger Nucleases (ZFNs). ZFNs have been described in U.S. Pat. No. 9,322,006, which is incorporated herein by reference in its entirety, and are well known in the art. ZFNs are synthetic proteins consisting of an engineered zinc finger DNA-binding domain fused to the cleavage domain of an endonuclease, for example, Fok1. ZFNs can be designed to cleave almost any long stretch of double-stranded DNA by the modification of the zinc finger DNA-binding domain. ZFNs form dimers from monomers composed of a non-specific DNA cleavage domain of FokI nuclease fused to a zinc finger array engineered to bind a target DNA sequence. The DNA-binding domain of a ZFN is typically composed of 3-4 zinc-finger arrays. The amino acids at positions −1, +2, +3, and +6 relative to the start of the zinc finger ∞-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art. The FokI nuclease domain requires dimerization to cleave DNA and therefore two ZFNs with their C-terminal regions are needed to bind opposite DNA strands of the cleavage site (separated by 5-7 nt). The ZFN monomer can cut the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by theory, because the DNA-binding specificities of zinc finger domains can in principle be re-engineered using one of various methods, customized ZFNs can be constructed to target nearly any gene sequence. Publicly available methods for engineering zinc finger domains include Context-dependent Assembly (CoDA), Oligomerized Pool Engineering (OPEN), and Modular Assembly.

Several embodiments relate to a method and/or composition provided herein comprising one or more, two or more, three or more, four or more, or five or more ZFNs directed to a target sequence as described herein. In another aspect, a ZFN provided herein is capable of generating a targeted DSB. In one aspect, vectors comprising polynucleotides encoding one or more, two or more, three or more, four or more, or five or more ZFNs are provided to a cell by transformation methods known in the art (e.g., without being limiting, viral transfection, particle bombardment, PEG-mediated protoplast transfection or Agrobacterium-mediated transformation).

### D. Meganucleases

In some embodiments, genome engineering of a plant as described herein may employ a meganuclease. Meganucleases, which are commonly identified in microbes, are unique enzymes with high activity and long recognition sequences (>14 nt) resulting in site-specific digestion of target DNA. Engineered versions of naturally occurring meganucleases typically have extended DNA recognition sequences (for example, 14 to 40 nt). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

In one aspect, a method and/or composition provided herein comprises one or more, two or more, three or more, four or more, or five or more meganucleases directed to a target sequence as described herein. In some embodiments, a meganuclease provided herein is capable of generating a targeted DSB. In some embodiments, vectors comprising polynucleotides encoding one or more, two or more, three or more, four or more, or five or more meganucleases are provided to a cell by transformation methods known in the art (e.g., without being limiting, viral transfection, particle bombardment, PEG-mediated protoplast transfection or Agrobacterium-mediated transformation)

### II. Site-Specific Genome Modification

Certain aspects of the disclosure relate to methods of modifying the genome of a plant using site-specific genome modification techniques. Exemplary site-specific genome modification encompasses any genome modification technique that employs an enzyme that can modify a nucleotide sequence in a sequence-specific manner. Site-specific genome modification enzymes include, but are not limited to, nucleases, endonucleases, recombinases, invertases, transposases, methytransferases, demethlylases, aminases, deaminases, helicases, and any combination thereof. In some embodiments, site-specific genome modification of an alfalfa plant as described herein may employ any site-specific genome modification enzyme. As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme is a recombinase. In some embodiments, a site-specific genome modification enzyme is a transposase. Site-specific genome modification enzymes include, but are not limited to, nucleases, endonucleases, recombinases, invertases, and transposases and the like.

In some embodiments, the site-specific genome modification enzyme is a recombinase. Non-limiting examples of recombinases include a tyrosine and serine recombinases and coupled with a DNA recognition motifs, for example, a Cre recombinase, a Gin recombinase, a Flp recombinase, and a Tnp1 recombinase. In another aspect, a serine recombinase coupled with a DNA recognition motif, for example, a PhiC31 integrase, an R4 integrase, and a TP-901 integrase. In an aspect, a recombinase is tethered to a zinc-finger DNA-binding domain, or a TALE DNA-binding domain, or a Cas9 nuclease.

The Flp-FRT site-directed recombination system comes from the 2μ plasmid from the baker's yeast Saccharomyces cerevisiae. In this system, Flp recombinase (flippase) recombines sequences between flippase recognition target (FRT) sites. FRT sites comprise 34 nucleotides. Flp binds to the “arms” of the FRT sites (one arm is in reverse orientation) and cleaves the FRT site at either end of an intervening nucleic acid sequence. After cleavage, Flp recombines nucleic acid sequences between two FRT sites.

Cre-lox is a site-directed recombination system derived from the bacteriophage P1 that is similar to the Flp-FRT recombination system. Cre-lox can be used to invert a nucleic acid sequence, delete a nucleic acid sequence, or translocate a nucleic acid sequence. In this system, Cre recombinase recombines a pair of lox nucleic acid sequences. Lox sites comprise 34 nucleotides, with the first and last 13 nucleotides (arms) being palindromic. During recombination, Cre recombinase protein binds to two lox sites on different nucleic acids and cleaves at the lox sites. The cleaved nucleic acids are spliced together (reciprocally translocated) and recombination is complete. In another aspect, a lox site provided herein is a loxP, lox 2272, loxN, lox 511, lox 5171, lox71, lox66, M2, M3, M7, or M11 site.

In another aspect, the site-specific genome modification enzyme is a dCas9-recombinase fusion protein. As used herein, a “dCas9-recombinase fusion protein” is a dCas9 with a protein fused to the dCas9 in such a manner that the recombinase is catalytically active on the DNA. In some embodiments, dCas9 may be fused with the catalytic domain of any enzyme such that the catalytic domain is catalytically active on DNA. In another aspect, a DNA transposase is attached to a DNA binding domain for example, a TALE-piggyBac and TALE-Mutator.

Several embodiments relate to promoting DNA recombination by providing a site-specific genome modification enzyme to a plant cell. In some embodiments, recombination is promoted by providing a strand separation inducing reagent. In one aspect, the site-specific genome modification enzyme is selected from an endonuclease, a recombinase, an invertase, a transposase, a helicase or any combination thereof. In some embodiments, recombination occurs between B chromosomes. In some embodiments, recombination occurs between a B chromosome and an A chromosome.

Several embodiments relate to promoting integration of one or more DNAs of interest by providing a site-specific genome modification enzyme. In some embodiments, integration of one or more DNAs of interest is promoted by providing a strand separation inducing reagent. In one aspect, the site-specific genome modification enzyme is selected from an endonuclease, a recombinase, a transposase, a helicase or any combination thereof. Any DNA sequence can be integrated into a target site of a chromosome sequence by introducing the DNA sequence and the provided site-specific genome modification enzymes. Any method provided herein can utilize any site-specific genome modification enzyme provided herein.

Several embodiments relate to a method and/or a composition provided herein comprising at least one, at least two, at least three, at least four, at least five, at least six, at least seven, at least eight, at least nine, or at least ten site-specific genome modification enzymes. In yet another aspect, a method and/or a composition provided herein comprises at least one, at least two, at least three, at least four, at least five, at least six, at least seven, at least eight, at least nine, or at least ten polynucleotides encoding at least one, at least two, at least three, at least four, at least five, at least six, at least seven, at least eight, at least nine, or at least ten site-specific genome modification enzymes.

### III. Plant Transformation Constructs

Transformation constructs, as used herein, may be a chimeric DNA molecule which is designed for introduction into a host cell by genetic transformation. Exemplary transformation constructs will comprise all of the genetic elements necessary to direct the expression of one or more exogenous nucleic acid sequences. Vectors used for plant transformation may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large genetic sequences comprising more than one selected gene.

Particularly useful for transformation are expression cassettes that have been isolated from such vectors. DNA segments used for transforming plant cells will, of course, generally comprise the cDNA, gene or genes which one desires to introduce into and have expressed in the host cells. These DNA segments can further include structures such as promoters, enhancers, polylinkers, or even regulatory genes as desired. The DNA segment or gene chosen for cellular introduction will often encode a protein which will be expressed in the resultant genetically modified cells resulting in a screenable or selectable trait and/or will impart an improved phenotype to the resulting genetically modified plant. However, this may not always be the case, and the disclosure also encompasses genetically modified plants incorporating non-expressed transgenes.

In accordance with the disclosure, a nucleic acid vector comprising a coding sequence may be introduced into a plant, such that, when the vector is transformed into a plant as described herein, the coding sequence is expressed in the plant. Expression of the coding sequence in the resulting genetically modified plant results in the plant exhibiting at least improved forage production or quality when compared to a plant lacking expression of the coding sequence.

In accordance with the disclosure, a genetically modified plant may be defined as a plant comprising at least one genetically modified cell. A genetically modified plant may be regenerated from a genetically modified cell or plant part comprising genetically modified cells, and thus the genetic modification may be heritable and inherited by progeny thereof. The progeny thereof that inherit the genetic modification are also considered genetically modified plants. A genetically modified plant, as used herein, also refers to a plant in which at least one genetically modified cell is introduced to a plant or arises as a result of genetic modification techniques directly applied to the plant. A genetically modified cell, as contemplated by the disclosure, may be a cell in which the endogenous genome has been genetically modified; a cell in which one or more exogenous, coding nucleic acids have been introduced that do not integrate into the genome, yet are capable of autonomous replication; or a combination thereof. The disclosure contemplates genetically modified cells produced by any genetic modification technique. A genetic modification technique as used herein encompasses any technique known to those in the art that can modify the genome of a cell including, but not limited to, genome editing, site-specific genetic recombination, epigenetic modifications, and genetic transformation.

As used herein, a “protein/Coding DNA molecule” or “polypeptide/Coding DNA molecule” refers to a DNA molecule comprising a nucleotide sequence that encodes a protein or polypeptide. A “coding sequence” or “protein/Coding sequence” or “polypeptide/Coding sequence” means a DNA sequence that encodes a protein or polypeptide. A “sequence” means a sequential arrangement of nucleotides or amino acids. The boundaries of a protein/Coding sequence or polypeptide/Coding sequence are usually determined by a translation start codon at the 5′-terminus and a translation stop codon at the 3′-terminus. A protein/Coding molecule or polypeptide/Coding molecule may comprise a DNA sequence encoding a protein or polypeptide sequence. As used herein, the terms “transgene expression,” “expressing a transgene,” “protein expression,” “polypeptide expression,” “expressing a protein,” and “expressing a polypeptide” refer to the production of a protein or polypeptide through the process of transcribing a DNA molecule into messenger RNA (mRNA) and translating the mRNA into polypeptide chains, which may be ultimately folded into proteins.

A DNA molecule may be operably linked to a heterologous promoter in a DNA construct for use in facilitating transcription in a cell transformed with a recombinant DNA molecule. As used herein, “operably linked” means two DNA molecules linked in manner so that one may affect the function of the other. Operably-linked DNA molecules may be part of a single contiguous molecule and may or may not be adjacent. For example, a promoter is operably linked with a DNA molecule in a DNA construct where the two DNA molecules are so arranged that the promoter may affect the transcription of a DNA molecule.

A transgene, as used herein, may be a segment of DNA which has been incorporated into a host genome or is capable of autonomous replication in a host cell and is capable of causing the transcription of one or more nucleic acid sequences, including an RNAi construct as described herein. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation which was modified with the DNA segment. As used herein, a “DNA construct” is a recombinant DNA molecule comprising two or more heterologous DNA sequences. DNA constructs are useful for transgene expression and may be comprised in vectors and plasmids. DNA constructs may be used in vectors for the purpose of genome modification, that is the introduction of heterologous DNA into a host cell, in order to produce genetically modified plants and cells, and as such may also be contained in the plastid DNA or genomic DNA of a genetically modified plant, seed, cell, or plant part. As used herein, a “vector” means any recombinant DNA molecule that may be used for the purpose of genetically modifying a plant or plant cell. Recombinant DNA molecules as set forth in the sequence listing, can, for example, be inserted into a vector as part of a construct having the recombinant DNA molecule operably linked to a promoter that functions in a plant to drive transcription. Methods for constructing DNA constructs and vectors are well known in the art. The components for a DNA construct, or a vector comprising a DNA construct, can include, but are not limited to, one or more of the following: a suitable promoter for the expression of an operably linked DNA, an operably linked DNA molecule to be transcribed, and a 3′ untranslated region (3′-UTR). Promoters useful in practicing the embodiments contemplated in the disclosure include those that function in a plant for expression of an operably linked polynucleotide. Such promoters are varied and well known in the art and include those that are inducible, viral, synthetic, constitutive, temporally regulated, spatially regulated, and/or spatio-temporally regulated. Additional components could include, but are not limited to, one or more of the following elements: 5′-UTR, enhancer, leader, cis-acting element, intron, chloroplast transit peptides (CTP), and one or more selectable marker transgenes.

A Heterologous promoter or sequence, as used herein, may refer to a promoter or a nucleic sequence which is not normally present in a given host genome in the genetic context in which that promoter or sequence is currently found. In this respect, the promoter or sequence may be from another species, organism, plant, tree, or variety, or may be native to the host genome, but be rearranged with respect to other genetic sequences within the host genome. For example, a regulatory sequence may be heterologous in that it is linked to a different sequence relative to the native regulatory sequence. In addition, a particular recombinant DNA molecule may be heterologous with respect to a cell or organism into which it is inserted when it would not naturally occur in that particular cell or organism.

Recombinant DNA molecules of the disclosure may be synthesized and modified by methods known in the art, either completely or in part, especially where it is desirable to provide sequences useful for DNA manipulation (such as restriction enzyme recognition sites or recombination-based cloning sites), plant-preferred sequences, or sequences useful for DNA construct design. The disclosure includes recombinant DNA molecules and proteins having at least about 80% (percent) sequence identity, about 81% sequence identity, about 82% sequence identity, about 83% sequence identity, about 84% sequence identity, about 85% sequence identity, about 86% sequence identity, about 87% sequence identity, about 88% sequence identity, about 89% sequence identity, about 90% sequence identity, about 91% sequence identity, about 92% sequence identity, about 93% sequence identity, about 94% sequence identity, about 95% sequence identity, about 96% sequence identity, about 97% sequence identity, about 98% sequence identity, about 99% sequence identity, or about 100% sequence identity to a sequence provided herein, for instance the sequences set forth as SEQ ID NOs: 1-10. As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar, Inc., 1228 S. Park St., Madison, Wis. 53715), and MUSCLE (version 3.6) (RC Edgar, Nucleic Acids Research (2004) 32(5):1792-1797) with default parameters. An “identity fraction” for aligned segments of a test sequence and a reference sequence is the number of identical components which are shared by the two aligned sequences divided by the total number of components in the reference sequence segment, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence.

In some embodiments, a sequence as described herein may be transcribed or expressed at any level in the plant such that it may be detected in the plant using techniques known in the art. An FT gene sequence may be expressed in a lower quantity in a genetically modified plant or variety than in a plant not engineered according to the invention. In some embodiments, FT gene expression is defined as reduced about 20, 30%, 40, 50%, 60%, 70%, 80%, 90%, 95% or 100% relative to a wild-type plant.

The DNA sequence between the transcription initiation site and the start of the coding sequence, i.e., the untranslated leader sequence, can also influence gene expression. One may thus wish to employ a particular leader sequence with a transformation construct of the disclosure. Useful leader sequences are contemplated to include those which comprise sequences predicted to direct optimum expression of the attached sequence, i.e., to include a consensus leader sequence which may increase or maintain mRNA stability and prevent inappropriate initiation of translation. The choice of such sequences will be known to those of skill in the art in light of the disclosure.

Transformation constructs prepared in accordance with the disclosure will typically include a 3′ end DNA sequence that acts as a signal to terminate transcription and allow for the poly-adenylation of the mRNA produced by coding sequences operably linked to a promoter. Non-limiting examples of terminators that are deemed to be useful for use in transgenic plants include those from the nopaline synthase gene of Agrobacterium tumefaciens (nos 3′ end) (Bevan et al., 1983), the terminator for the T7 transcript from the octopine synthase gene of Agrobacterium tumefaciens, and the 3′ end of the protease inhibitor I or II genes from potato or tomato.

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait which one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). Many examples of suitable marker proteins are known to the art and can be employed in the practice of the disclosure. Examples include, but not limited to, neo, bar, bxn; a mutant acetolactate synthase (ALS), a methotrexate resistant DHFR, β-glucuronidase (GUS); R-locus, β-lactamase, xylE, α-amylase, tyrosinase, β-galactosidase, luciferase (lux), aequorin, and green fluorescent protein (PCT Publication No. WO/1997/41228, which is incorporated herein by reference in its entirety).

Included within the terms “selectable” or “screenable” markers also are genes which encode a “secretable marker” whose secretion can be detected as a means of identifying or selecting for genetically modified cells. Examples include markers which are secretable antigens that can be identified by antibody interaction, or even secretable enzymes which can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR-S).

### IV. Antisense and DNA-Directed RNA Interference Constructs

In specific embodiments of the disclosure, endogenous gene activity can be down-regulated by any means known in the art, including through the use of an antisense polynucleotide or polynucleotide molecule targeted through an RNA interference pathway to an mRNA that is encoded by an endogenous FT gene. In particular, DNA constructs comprising a gene sequence, including fragments thereof, in antisense orientation, or combinations of sense and antisense orientation, may be used to decrease or effectively eliminate the expression of the FT gene in a plant. In specific embodiments, constructs comprising sequences both in antisense and sense orientation may be designed so that the three dimensional structure of the RNA molecule encoded by the construct facilitates the respective antisense and sense sequences within the encoded RNA molecule hybridizing to form double-stranded RNA, for example, a short hairpin RNA (shRNA) vector. Accordingly, these constructs may be used to partially “knockdown” or completely “knockout” the function of the coding sequence or homologous sequences thereof.

Techniques that take advantage of cellular RNAi pathways to silence the expression of genes are well known in the art. These techniques are based on the fact that a double stranded RNA molecule can direct the degradation or prevent the translation of other RNA molecules, for example, pre-mRNAs and mRNAs, that comprise nucleotide sequences that are complementary to either strand of the double stranded RNA molecule. For example, the expression levels of the mRNA encoded by a coding sequence and the polypeptide translated therefrom can therefore be down-regulated by expression of a fragment or a longer portion of the coding sequence in sense, antisense, or both orientations.

Antisense silencing methodologies, and in some aspects cellular RNAi pathways, take advantage of the fact that nucleic acids tend to pair and hybridize with “complementary” sequences. Complementary, as used herein, refers to polynucleotides that are capable of base-pairing according to the standard Watson/Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common nucleotide bases such as inosine, 5-methylcytosine, 6-methyladenine, hypoxanthine and others in hybridizing sequences does not interfere with pairing.

Targeting double stranded (ds) DNA with a polynucleotide leads to triple-helix formation, and targeting single stranded RNA with the same will lead to double-helix formation. Antisense oligonucleotides, when introduced into a target cell, specifically bind to their target polynucleotide and interfere with transcription, RNA processing, transport, translation and/or stability. Antisense and DNA-directed RNAi (ddRNAi) constructs, or DNA encoding such RNA's, may be employed to inhibit gene transcription or translation or both within a host cell, either in vitro or in vivo. In certain embodiments of the disclosure, such an oligonucleotide may comprise any unique portion of a nucleic acid sequence provided herein. In certain embodiments of the disclosure, such a sequence comprises at least 18, 30, 50, 75, 100, 150, or 200 or more contiguous base pairs of the nucleic acid sequences provided herein, which may be in sense and/or antisense orientation. By including sequences in both sense and antisense orientation, increased suppression of the corresponding coding sequence may be achieved. By including sequences in both sense and antisense orientation such that the three dimensional structure facilitates their hybridization in double-stranded RNA may further increase the suppression of the corresponding coding sequence.

Constructs may be designed that are complementary to all or part of the promoter and other control regions, exons, which include the 5′ and 3′ UTRs, introns, or exon-intron boundaries of a gene, or any portion of an ncRNA. It is contemplated that the most effective constructs may include regions complementary to intron/exon splice junctions. Thus, it is proposed that an embodiment includes a construct with sequences complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base pair mismatches. For example, sequences of fifteen base pairs in length may be termed complementary when they have complementary nucleotides at thirteen or fourteen base pairs. Naturally, sequences which are completely complementary will be sequences which are entirely complementary throughout their entire length and have no base pair mismatches. Other sequences with lower degrees of homology also are contemplated. For example, an RNAi or antisense construct which has limited regions of high homology, but also contains a non-homologous region, e.g., as in a ribozyme, could be designed. Methods for selection and design of sequences that are processed through an RNAi pathway are well known in the art. These molecules, though having less than 50% homology, would bind to target sequences under appropriate conditions.

It may be advantageous to combine portions of genomic DNA with cDNA or synthetic sequences to generate specific ddRNAi constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

Another embodiment contemplates plants that are genetically modified such that expression of an endogenous non-coding RNA (ncRNA) that is capable of down-regulating expression of an endogenous gene through an RNA interference pathway, for example, microRNAs (miRNAs), endogenous short interfering RNAs (endo-siRNAs), and piwi-interacting RNAs (piRNAs), is either activated or increased. In particular embodiments, a promoter may be integrated into genome of a plant such that it operably drives expression of an ncRNA. In other embodiments, the modification down-regulates or increases the expression of a repressive or activating trans-acting regulatory factor of an ncRNA, or disrupts a suppressive cis-regulatory element thereof. Accordingly, this may be used to partially “knockdown” or completely “knockout” the function of a gene, coding sequence, or ncRNA complementary to the ncRNA.

### V. Methods for Genetic Transformation

The disclosure contemplates methods of genetically modifying cells that employ genetic transformation techniques as well as the genetically modified cells produced using those techniques. Genetic transformation, as used herein, refers to any process of introducing a DNA sequence or construct, e.g., a vector or expression cassette, into a cell or protoplast in which all or a portion of the exogenous DNA is incorporated into a chromosome or is capable of autonomous replication. Suitable methods for transformation of plant or other cells for use with the embodiments of the disclosure are believed to include virtually any method by which DNA can be introduced into a cell, such as by direct delivery of DNA such as by PEG-mediated transformation of protoplasts, by desiccation/inhibition-mediated DNA uptake, by electroporation (U.S. Pat. No. 5,384,253, specifically incorporated herein by reference in its entirety), by Agrobacterium-mediated transformation (U.S. Pat. Nos. 5,591,616 and 5,563,055; both specifically incorporated herein by reference) and by acceleration of DNA coated particles (U.S. Pat. Nos. 5,550,318; 5,538,877; and 5,538,880; each specifically incorporated herein by reference in its entirety), etc. Through the application of techniques such as these, the cells of virtually any plant species may be stably transformed, and these cells developed into genetically modified plants.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by U.S. Pat. No. 5,563,055, specifically incorporated herein by reference in its entirety.

Another method for delivering transforming DNA segments to plant cells in accordance with the disclosure is microprojectile bombardment (U.S. Pat. Nos. 5,550,318; 5,538,880; 5,610,042; and PCT Publication No. WO/1994/09699; each of which is specifically incorporated herein by reference in its entirety). In this method, particles may be coated with nucleic acids and delivered into cells by a propelling force.

### VI. Production and Characterization of Genetically Modified Plants

After effecting delivery of exogenous DNA to recipient cells, the next steps generally concern first identifying and selecting the transformed cells and from those cells identifying the selecting the genetically modified cells for further culturing and plant regeneration. An exemplary transformed cell is one in which the genome has been altered by the introduction of an exogenous DNA molecule into that cell, and an exemplary processes of regeneration broadly encompass growing a plant from a plant cell, for example, a plant protoplast, callus, or explant.

In order to improve the ability to identify transformed and genetically modified cells contemplated by the invention, one may desire to employ one or more selectable or screenable marker genes with a transformation vector prepared in accordance with the disclosure. In this case, one would then generally assay the potentially transformed and modified cell population by exposing the cells to a selective agent or agents, or one would screen the cells for the desired marker gene trait. It is believed that DNA is introduced into only a small percentage of target cells in any one study. In order to provide an efficient system for identification of those cells that are transformed and predisposed to genetic modification one may employ a means for selecting those cells that are stably transformed. One exemplary embodiment of such a method is to introduce, into the host cell, a marker gene which confers resistance to some normally inhibitory agent, such as an antibiotic or herbicide. Potentially transformed cells then are exposed to the selective agent. In the population of surviving cells will be those cells where, generally, the resistance/Conferring gene has been integrated and expressed at sufficient levels to permit cell survival. Cells may be tested further to confirm stable integration of the exogenous DNA.

Cells that survive the exposure to the selective agent, or cells that have been scored positive in a screening assay, may then be selected again using a second, distinct selection paradigm that detects those cells that contain the genetic modification. Cells that survive the exposure to the second selective agent, or cells that have been scored positive in the second screening assay, may be cultured in media that supports regeneration of plants. The genetically modified cells, identified by selection or screening and cultured in an appropriate medium that supports regeneration, will then be allowed to mature into plants.

To confirm the presence of the genetic modification in the regenerating plants, a variety of assays may be performed. Such assays include, for example, “molecular biological” assays, such as Southern and northern blotting and polymerase chain reaction (PCR); “biochemical” assays, such as detecting the absence or presence of a protein product, e.g., by immunological means (ELISAs and western blots) or by enzymatic function; plant part assays, such as leaf or root assays; and also, by analyzing the phenotype of the whole regenerated plant.

### VII. Breeding Plants of the Disclosure

In addition to direct transformation of a particular plant genotype with a construct prepared according to the disclosure, genetically modified plants may be made by crossing a plant having a selected genetic modification of the disclosure to a second plant lacking the construct. For example, a selected coding sequence can be introduced into a particular plant variety by crossing, without the need for ever directly transforming a plant of that given variety. Therefore, the disclosure not only encompasses a plant directly modified or regenerated from cells which have been modified in accordance with the disclosure, but also the progeny of such plants.

As used herein the term “progeny” denotes the offspring of any generation of a parent plant prepared in accordance with the disclosure, wherein the progeny comprises a selected DNA construct. “Crossing” a plant to provide a plant line having one or more added transgenes relative to a starting plant line, as disclosed herein, is exemplified by the techniques that result in a coding sequence of the disclosure being introduced into a plant line by crossing a starting line with a donor plant line that comprises a first selected DNA of the disclosure. To achieve this in a plant one could, for example, perform the following steps:
(a) plant seeds of the first (starting line) and second (donor plant line that comprises a first selected DNA of the disclosure) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

An example of the backcrossing methods that are contemplated by the disclosure includes, but is in no way limited to, the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

The traditional backcross technique can be adjusted to include more than one recurrent parent when breeding plants such as alfalfa, in which homozygosity can result in inbreeding depression, agronomic performance decline, and loss of traits of interest. This type of backcrossing is known as modified backcrossing and employs at least two different recurrent parents to produce a sufficiently heterozygous population with the agronomically significant characteristics of the recurrent parents and the trait or traits of interest from the donor parent. Modified backcrossing may also be used to replace the original recurrent parent with one or more distinct parents to stack different characteristics from each, and therefore providing additional improvement over a single recurrent parent.

Exemplary introgressions of a DNA element into a plant genotype contemplated by this disclosure are the results of the process of a backcross conversion or modified backcross conversion. A plant genotype into which a DNA sequence has been introgressed may be referred to as a backcross or modified backcross converted genotype, line, inbred, or hybrid. Similarly a plant genotype lacking the desired DNA sequence may be referred to as an unconverted genotype, line, inbred, or hybrid.

In some embodiments, asexual reproduction or propagation may be used to obtain a progeny plant in accordance with the disclosure. Techniques to achieve asexual propagation or reproduction in the plant may include, for example, grafting, budding, top-working, layering, runner division, cuttings, rooting, T-budding, and the like.

### VIII. Definitions

Acid detergent fiber (ADF): Percentage of fiber that is soluble (dissolves) in weak acid. ADF is used to calculate digestibility. A measurement that approximates the amount of cellulose fiber and ash present in a feed. Forages with high ADF values are less digestible than forages with low ADF values, and therefore provide fewer nutrients to the animal through digestion. Because of this relationship, ADF serves as an estimate of digestibility and can be used by nutritionists to predict the energy that will be available from a forage.

Acid detergent lignin (ADL): The amount of acid detergent lignin.

Alfalfa Vigor: A metric that reflects the overall health and strength of an alfalfa plant. Plants receive a general assessment of their appearance that includes evaluations of their overall health, fitness, and presentation of any phenotypes of interest. A higher score indicates a plant with higher overall health and strength.

Crude protein (CP): A measurement of the total nitrogen concentration in a forage. This technique measures not only the nitrogen present in true proteins, but also that present in non-protein forms such as ammonia, urea, and nitrate. Because most of the non-protein forms of nitrogen are converted to true protein by the rumen microorganisms, CP is considered by nutritionists to provide an accurate measure of the protein that will be available to ruminant animals from a given forage.

Down-regulated: As used herein, down-regulation of, for example, a gene, ncRNA, transgene, pre-mRNA, mRNA, or polypeptide, refers to a reduction in its expression or its downstream product, e.g., the polypeptide produced by a gene, whether by natural means or as a result of genetic modification.

Dry weight: The weight of a plant matter sample after it has been completely dried, i.e., the weight of the dry matter contained within a plant matter sample.

Dietary Dry Matter (DM): The matter e.g., protein, fiber, fat, minerals, etc., within a sample of alfalfa excluding water. It is one metric by which yield may be calculated.

Expression: The combination of intracellular processes, including transcription and translation undergone by any DNA molecule to produce a polypeptide or a functional nucleic acid, e.g., a mRNA, and ncRNA, antisense molecule, ribozyme, aptamer, etc.).

Fresh weight: The weight of a plant matter sample recorded immediately after it has been harvested.

Neutral detergent fiber percent (NDF): A measurement that represents the total amount of fiber present in the alfalfa. Because fiber is the portion of the plant most slowly digested in the rumen, it is this fraction that fills the rumen and becomes a limit to the amount of feed an animal can consume. The higher the NDF concentration of a forage, the slower the rumen will empty, which reduces what an animal will be able to consume. NDF is therefore used by nutritionists as an estimate of the quantity of forage that an animal will be able to consume. Forages with high NDF levels can limit intake to the point that an animal is unable to consume enough feed to meet their energy and protein requirements.

Neutral detergent fiber digestibility percent (NDFD): The percentage of the a neutral detergent fiber within a plant matter sample that can be digested.

Overexpress: As used herein, “overexpress” refers to increased expression of a gene, coding sequence, ncRNA, mRNA, or polypeptide over that found in nature or a control plant or tissue. In some embodiments, “overexpress” may refer to greater expression of a gene, coding sequence, ncRNA, mRNA, or polypeptide in a genetically modified plant, when compared to a plant lacking the genetic modification.

As used herein, the term “a” or “an” may refer one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

### EXAMPLES

### Example 1

Generation of transgenic MsFT knockdown alfalfa lines that exhibit delayed flowering and increased branching.

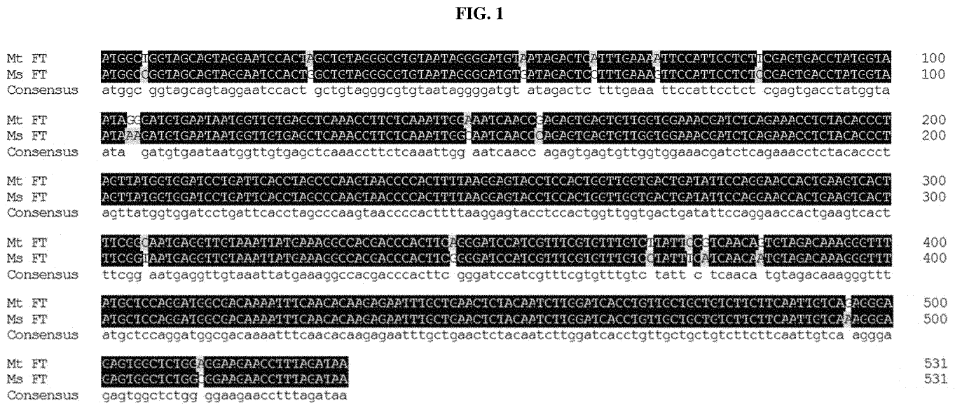

The M. sativa FT gene (MsFT) was identified and is included herein as SEQ ID NO:1, and its full-length coding sequence, SEQ ID NO:2, was generated. Alignment analysis of the full-length MsFT coding sequence revealed that it is 96.43% similar to the full-length M. truncatula FT (MtFT) coding sequence, SEQ ID NO:10 (FIG. 1), which indicates that the two genes are highly conserved.

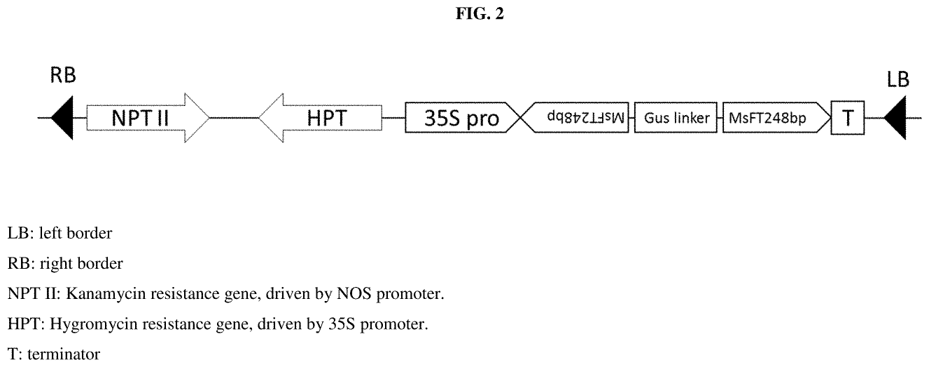

An RNA interference construct designed to knockdown endogenous MsFT expression was generated. A 248 bp portion of the MsFT coding sequence, which includes the highly conserved segment B region, was amplified using primers SEQ ID NOs:8 and 9, in both the sense and antisense directions, SEQ ID NOs:4 and 5. The amplified sense and antisense fragments were then cloned into a pENTR™/D-TOPO® entry vectors and subsequently transferred using GATEWAY® Technology LR recombination reactions into the pANDA35HK binary plant transformation vector to yield the MsFT RNA knockdown vector pANDA35HK-MsFT-RNAi:FT242. In particular, the two LR Clonase™ cassettes in the pANDA35HK vector, which flank a 920 bp β-glucuronidase (GUS) reporter cassette, were recombined with the sense and antisense MsFT fragments, respectively, which placed these fragments under the control of an enhanced Cauliflower mosaic virus (35SCaMV) promoter. The relative position and directionality of this engineered MsFT RNAi cassette and other notable components within the T-DNA segment of the pANDA35HK-MsFT-RNAi:FT242 construct are shown in FIG. 2.

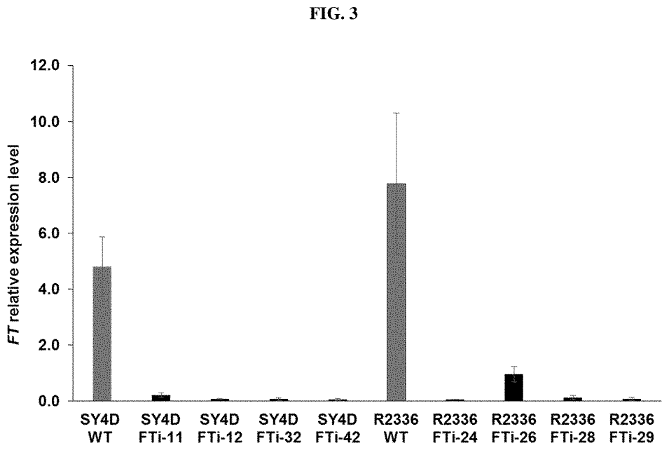

The pANDA35HK-MsFT-RNAi:FT242 construct was introduced into Agrobacterium strain EHA105 using a freezing/heat-shock method. Alfalfa lines R2336 and Regen SY4D were transformed separately by these Agrobacterium via somatic embryogenesis and the standard leaf disk protocol. Forty-nine transgenic lines derived from the Regen SY4D background and 35 transgenic lines derived from the R2336 background were produced. Analysis of MsFT transcript expression in each transgenic line was performed using total RNA that was extracted from fully-expanded, mature leaves. The total RNA was reverse transcribed to make cDNA, and RT-PCR was performed using that cDNA. Expression analysis results for 8 exemplary transgenic MsFT knockdown alfalfa lines are displayed in FIG. 3. All 8 exemplary lines exhibited MsFT transcript levels that were decreased by more than 80% when compared to those of wildtype plants of the corresponding background. Of the 8 exemplary lines, 4 were derived from each of the Regen SY4D and R2336 backgrounds as shown in Table 1. Plants from these exemplary 8 lines were transitioned to soil and maintained under the following greenhouse conditions: 24° C. at day/22° C. at night, 16 h/8 h photoperiod, and relative humidity of 70%.

TABLE 1
Exemplary transgenic MsFT knockdown lines.

| Transgenic MsFT knockdown alfalfa line | |
|---|---|
| identification number | Background |
| FTi-11 | Regen SY4D |
| FTi-12 | Regen SY4D |
| FTi-32 | Regen SY4D |
| FTi-42 | Regen SY4D |
| FTi-24 | R2336 |
| FTi-26 | R2336 |
| FTi-28 | R2336 |
| FTi-29 | R2336 |

The flowering time of the 8 exemplary lines was compared to that of the wildtype controls. Alfalfa has multiple developmental stages including an initial vegetative stage. When environmental conditions are correct, this vegetative stage is followed, in order, by a bud stage, flowering stage, and a seed development stage. The vegetative stage is further divided into early, mid, and late periods based on objective morphological standards; and the budding and flowering stages are each further divided into early and late periods also based on objective morphological characteristics. An alfalfa plant may also be characterized as at “first flower,” “full-flower,” and “postflower.” In the case of the present analysis, each plant was monitored starting at the same growth stage until the first fully open flower was observed. The duration of time until that first fully open flower was recorded for each plant as its flowering time.

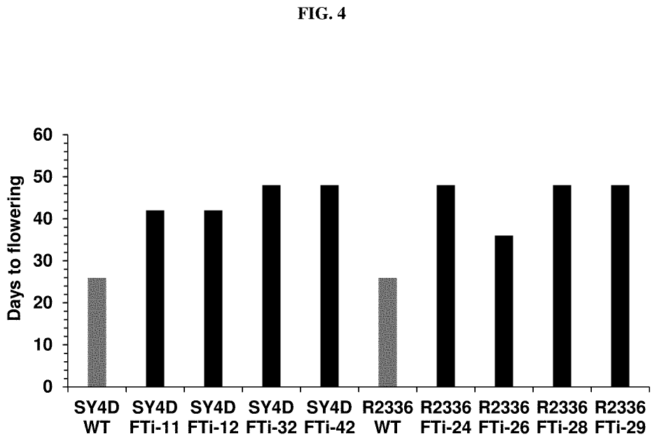

The plants of the 4 Regen SY4D-derived transgenic lines exhibited a delay in flowering time that ranged from 16 to 22 days when compared with wildtype Regen SY4D plants; and the plants of the 4 R2336-derived transgenic lines exhibited a delay in flowering time that was typically 22 days when compared with wildtype R2336 plants (FIG. 4). The duration of the flowering delays observed for each transgenic line positively correlated with the extent to which the MsFT transcript was decreased (FIG. 3 and FIG. 4). These 8 exemplary transgenic MsFT knockdown lines were also vegetatively propagated from the T₀ plants using shoot cuttings. The delayed flowering phenotype was found to be stable in all of the vegetatively propagated plants that were derived from any of these 8 exemplary lines.

Unexpectedly, plants of the exemplary transgenic MsFT knockdown lines also produced more branches when compared against wildtype plants of the corresponding background at a time point after the corresponding wildtype plants had begun to flower (Tables 2 and 3).

TABLE 2
The average number of branches on plants of the exemplary Regen
SY4D-derived transgenic MsFT knockdown lines at a time point
after the corresponding wildtype control plants exhibited a first fully
open flower.

| Line ID | Average Branch Number |
|---|---|
| WT | 4.3 ± 0.5 |
| FTi-11 | 7.3 ± 0.5 |
| FTi-12 | 6.7 ± 0.5 |
| FTi-32 | 7.0 ± 0.8 |
| FTi-42 | 7.3 ± 1.2 |

TABLE 3
The average number of branches on plants of the exemplary
R2336-derived transgenic MsFT knockdown lines at a time
point after the corresponding wildtype control plants exhibited
a first fully open flower.

| Line ID | Average Branch Number |
|---|---|
| WT | 4.7 ± 0.5 |
| FTi-24 | 7.0 ± 0.8 |
| FTi-26 | 5.0 ± 0.8 |
| FTi-28 | 7.3 ± 0.5 |
| FTi-29 | 6.7 ± 0.5 |

### Example 2

Controlled Greenhouse Trials Demonstrate that the Transgenic MsFT Knockdown Alfalfa Lines Exhibit Improved Forage Yield and Quality.

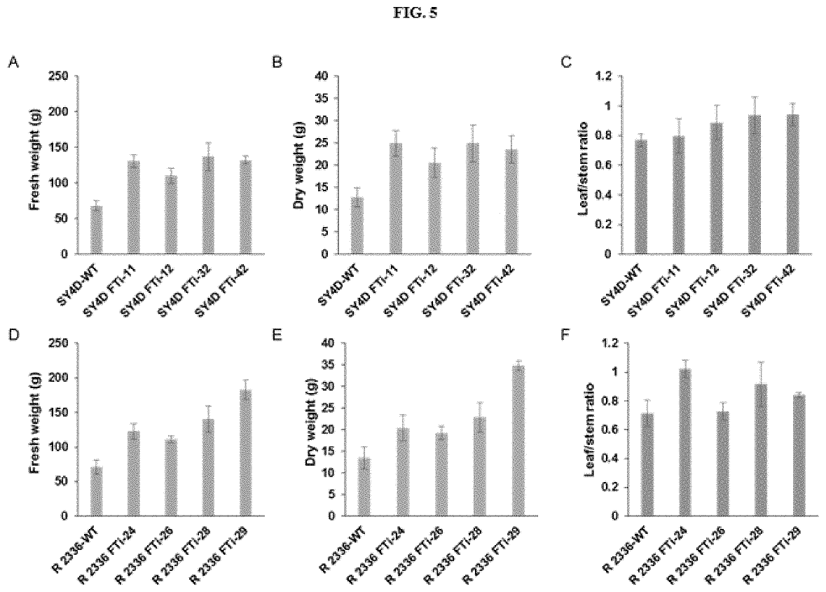

To measure the effect MsFT knockdown had on forage yield and quality, plants from the exemplary transgenic MsFT knockdown lines and wildtype Regen SY4D and R2336 plants were grown in a greenhouse with full nutrition under the following conditions: 24° C. at day/22° C. at night, 16 h/8 h photoperiod, and relative humidity of 70%. The plants were harvested at five weeks of age, when the wildtype alfalfa plants were in the early bloom stage, and fresh weight was measured. The biomass was then dried completely in an oven at 50° C. for 7 days, after which the dry weight was measured along with the leaf:stem ratio. The plants harvested from the four Regen SY4D-derived transgenic lines exhibited a 62%-101% increase in fresh weight, a 60%-94% increase in dry weight, and a 3%-22% increase in leaf:stem ratio when compared with harvested wildtype Regen SY4D plants (FIG. 5A, FIG. 5B, and FIG. 5C). The plants harvested from the four R2336-derived transgenic lines exhibited a 55%-157% increase in fresh weight, a 40%-157% increase in dry weight, and a 2%-43% increase in leaf:stem ratio when compared with harvested wildtype R2336 plants (FIG. 5D, FIG. 5E, and FIG. 5F).

To evaluate the forage quality of the dried harvests, they were ground with a Thomas® Wiley Mill using a 1.0 mm sieve. Near-infrared reflectance spectroscopy was performed on the ground harvests using a FOSS NIRS™ 5000/6500 Feed and Forage Analyser scanning at range of 1,100 nm to 2,500 nm. Each sample was scanned 8 times and the average spectra were used for calibration. The mathematical and statistical treatments of all spectra were performed with WinISI™ III Calibration Development Software, and the precision of NIRS has been assessed by regression analysis of the predicted values and actual determined values. The existing commercial NIRS prediction equations (07AHY50) developed by the NIRS Forage and Feed Testing Consortium were employed to estimate the following forage quality characteristics of the harvests: crude protein percent (CP), acid detergent fiber (ADF) percent, and acid detergent lignin (ADL) percent. All data were analyzed using the SAS GLM procedure (SAS Institute).

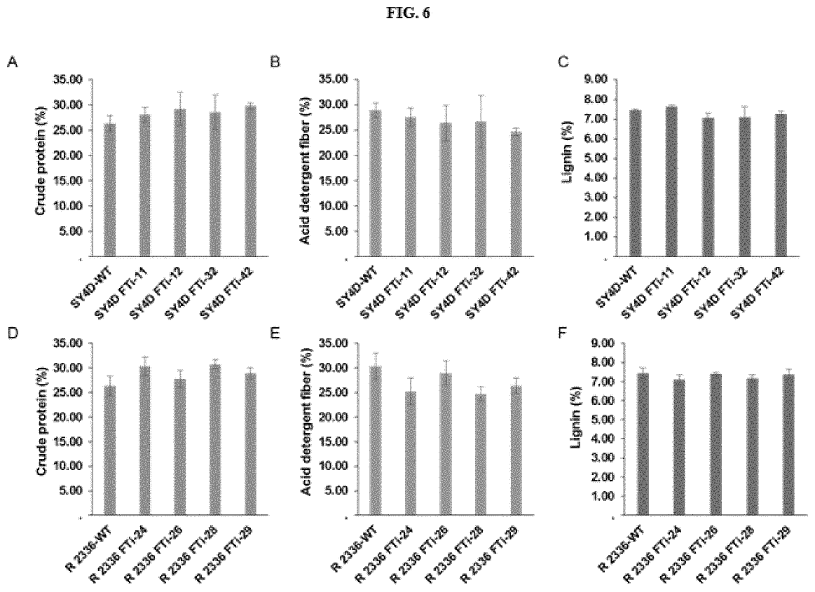

CP reflects the collective amount of true polypeptides, individual amino acids, nitrate, and non-protein nitrogen within the forage, and thus is a crucial indicator for assessing forage quality. The biomass harvested from the four Regen SY4D-derived transgenic lines and the four R2336-derived transgenic lines exhibited a 6%-13% and 5%-15% increase in CP, respectively, when compared against that of the corresponding wildtype control plants (FIG. 6A and FIG. 6D). ADF, on the other hand, reflects the relative amount of cellulose and lignin within the forage, and is inversely correlated with the digestibility of a forage. Measuring ADF and ADL percent in tandem can therefore further inform the factors affecting the indigestibility of a forage. The biomass harvested from the four Regen SY4D-derived transgenic lines and the four R2336-derived transgenic lines exhibited up to 15% and 18% reductions in ADF, respectively, when compared against that of the corresponding wildtype control plants (FIG. 6B and FIG. 6E). However, no significant changes were observed in lignin content (FIG. 6C and FIG. 6F).

Considering the CP and ADF data together, the forage produced from the 5-week-old exemplary transgenic knockdown lines was more digestible and of a higher nutritive quality than that produced from the 5-week-old wildtype controls. Further, the ADL data would suggest that in a greenhouse environment the increased digestibility of forage produced from the 5-week-old exemplary transgenic knockdown lines was not due to a substantial decrease in lignin content. Nonetheless, it is recognized that greenhouse testing such as that used here may not adequately assess some lignin biosynthetic pathways, as greenhouses typically eliminate many of the external stimuli that can modulate lignin biosynthesis within plants, e.g. wind, herbivory, etc. Therefore, the ADL data from these greenhouse trails does not preclude that RNAi knockdown of MsFT may also alter lignin biosynthesis pathways that cannot be accurately assessed in a typical greenhouse environment.

### Example 3

Field Trials Demonstrate that the Transgenic MsFT Knockdown Lines have Improved Forage Yield and Quality.

Field trials were performed to evaluate the agronomic performance of transgenic MsFT knockdown lines. The field trials were performed in West Salem, Wis., in 3 replicate plots for each line, except line FTi-28 for which only 2 replicate plots were used. Plot size was 76 ft. by 28 ft. There was 15 in. of space between each plant within a row. There was 30 in. of space between each range. Plots were managed under optimal forage production practices, and insecticide and herbicide applications were documented.

Each replicate for each line reflects four transplants, and all transplants were prepared in a greenhouse until transplanted. Each transgenic and wildtype line was propagated via vegetative stem cutting, and plants were watered and fertilized to optimize vigorous vegetative growth until time of transplanting in the field. Plant tops were clipped to 2.0 in. stubble height before transplanting to minimize planting stress.

All plants were transplanted to the field on Day 1. Plants were clipped back on Day 44, and vigor notes were taken on Day 64. Plant flowering notes started on Day 64, and were taken again on Days 67, 70, 72, 74, 77, and 79. Whole plant quality samples were also taken from each plot on Day 79 and the plots were, again, clipped back. A second set of vigor notes was taken on Day 93.

The agronomic vigor notes rated the plants on a scale from “1” to “9,” in which “1,” “5,” and “9” correspond to “poor,” “average,” and “excellent,” respectively. The vigor notes taken on Days 64 and 93 for the Regen SY4D-derived and R2336-Derived transgenic MsFT knockdown lines have been averaged together and are presented in Tables 4 and 5, respectively, along with that of the corresponding wildtype controls. The transgenic MsFT knockdown lines derived from either background exhibit improved vigor when compared to that of the corresponding wildtype control.

The flowering notes captured the number of plants per plot of four plants that had one or more representative stems with one or more fully opened florets. The flowering notes taken within a single day the for Regen SY4D-derived and R2336-Derived transgenic MsFT knockdown lines have been averaged together and are presented for each day in Tables 4 and 5, respectively, along with that of the corresponding wildtype controls. The transgenic MsFT knockdown lines derived from either background exhibit delayed flowering when compared to that of the corresponding wildtype control.

TABLE 4
Average vigor and number of plants flowering per plot for the exemplary Regen
SY4D-derived transgenic MsFT knockdown lines.

| | Avg. Vigor | Avg. number of plants flowering per plot | | | | | | |
|---|---|---|---|---|---|---|---|---|
| Line ID | Days 64 and 93 | Day 64 | Day 67 | Day 70 | Day 72 | Day 74 | Day 77 | Day 79 |
| WT | 5.0 | 0.0 | 1.0 | 3.0 | 3.3 | 3.7 | 4.0 | 4.0 |
| FTi-11 | 5.8 | 0.0 | 0.0 | 0.0 | 0.0 | 0.0 | 0.0 | 0.0 |
| FTi-12 | 6.0 | 0.0 | 0.0 | 0.0 | 0.0 | 0.0 | 0.0 | 0.0 |
| FTi-32 | 6.7 | 0.0 | 0.0 | 0.0 | 0.0 | 0.0 | 0.0 | 0.0 |
| FTi-42 | 7.0 | 0.0 | 0.0 | 0.0 | 0.0 | 0.0 | 0.0 | 0.0 |

TABLE 5
Average vigor and average number of plants flowering per plot for the exemplary
R2336-derived transgenic MsFT knockdown lines.

| | Avg. Vigor | Avg. number of plants flowering per plot | | | | | | |
|---|---|---|---|---|---|---|---|---|
| Line ID | Days 64 and 93 | Day 64 | Day 67 | Day 70 | Day 72 | Day 74 | Day 77 | Day 79 |
| WT | 5.0 | 0.0 | 0.0 | 2.0 | 2.7 | 3.7 | 4.0 | 4.0 |
| FTi-24 | 6.0 | 0.0 | 0.0 | 0.0 | 0.0 | 0.0 | 0.0 | 0.0 |
| FTi-26 | 5.5 | 0.0 | 0.0 | 0.3 | 0.7 | 0.7 | 1.3 | 2.0 |
| FTi-28 | 6.5 | 0.0 | 0.0 | 0.0 | 0.0 | 0.0 | 0.0 | 0.0 |
| FTi-29 | 6.3 | 0.0 | 0.0 | 0.0 | 0.0 | 0.0 | 0.0 | 0.5 |

When the plants were sampled on Day 79, they were cut 6 cm-8 cm above the surface of the soil. The harvested samples were dried at 55° C. in a forced air oven until they reached 92%-93% DM. These dried samples were then first ground through a Thomas® Wiley Mill using a 6.0 mm sieve and then reground through a UDY Cyclone Sample Mill using a 1.0 mm sieve. Near-infrared reflectance spectroscopy (NIRS) was performed on the ground harvests using a FOSS NIRS™ DS2500 F. The existing commercial NIRS prediction equations (07AHY50) developed by the NIRS Forage and Feed Testing Consortium were employed to estimate the following forage quality characteristics of the harvests: ADF, ADL, CP, dietary dry matter percent, neutral detergent fiber percent (NDF), and neutral detergent fiber digestibility percent (NDFD). For each of these characteristics, the measurements have been averaged for each Regen SY4D-derived and R2336-Derived transgenic MsFT knockdown lines over all replicates and are presented in Tables 6 and 7, respectively, along with that of the corresponding wildtype controls.

TABLE 6
Estimated forage quality of the exemplary Regen
SY4D-derived transgenic MsFT knockdown lines.

| Line ID | % ADF | % ADL | % CP | % DM | % NDF | % NDFD |
|---|---|---|---|---|---|---|
| WT | 27.81 | 5.11 | 20.16 | 94.26 | 34.30 | 46.64 |
| FTi-11 | 20.39 | 3.47 | 20.90 | 93.88 | 26.39 | 55.76 |
| FTi-12 | 21.06 | 3.41 | 21.96 | 93.85 | 26.55 | 56.02 |
| FTi-32 | 20.12 | 3.41 | 21.92 | 93.84 | 26.20 | 55.43 |
| FTi-42 | 23.79 | 4.07 | 23.25 | 94.09 | 29.34 | 52.66 |

TABLE 7
Estimated forage quality of the exemplary R2336-derived
transgenic MsFT knockdown lines.

| Line ID | % ADF | % ADL | % CP | % DM | % NDF | % NDFD |
|---|---|---|---|---|---|---|
| WT | 25.96 | 4.62 | 21.57 | 94.17 | 31.99 | 48.76 |
| FTi-24 | 20.52 | 3.39 | 23.12 | 93.94 | 25.93 | 56.19 |
| FTi-26 | 24.04 | 4.31 | 21.14 | 94.12 | 30.13 | 50.28 |
| FTi-28 | 21.34 | 3.64 | 21.74 | 93.97 | 27.12 | 55.16 |
| FTi-29 | 22.44 | 3.89 | 20.38 | 93.87 | 28.88 | 51.08 |

The transgenic MsFT knockdown lines derived from either background exhibit decreased ADF and ADL when compared to that of the corresponding wildtype controls. This indicates that the transgenic lines have improved digestibility that is at least partially due to decreased lignin content. In view of the greenhouse ADL data, this field ADL data was unexpected, but one of skill in the art would recognize that field growth exposes plants to external stimuli that are difficult to replicate in greenhouse trials, some of which may modulate lignification. In line with ADF and ADL metrics, NDF was also decreased in the transgenic MsFT knockdown lines derived from either background in comparison to that of the corresponding wildtype controls. NDF represents the total amount of fiber in a forage, and decreased NDF like those exhibited by the transgenic MsFT knockdown lines indicates the lines produce a forage that is more digestible as well as more consumable. Further, NDFD specifically reflects the rate at which the fiber fraction of the forage will be digested in the rumen. The transgenic MsFT knockdown lines derived from either background exhibit increased NDFD when compared to that of the corresponding wildtype controls. Therefore the fiber component of the forage produced by these transgenic lines is also more digestible. Lastly, the Regen SY4D-Derived transgenic MsFT knockdown lines exhibit increased CP when compared to that of the wildtype Regen SY4D controls, which is indicative of improved nutritional quality.

The references cited herein, to the extent that they provide exemplary procedural or other details supplementary to those set forth herein, are specifically incorporated herein by reference.

What is claimed is:

1. An engineered alfalfa plant comprising a modification in its genome that results in artificially down-regulated expression of the FLOWERING LOCUS T (FT) gene, wherein the modification confers to said plant improved forage production, quality, or digestibility, or a combination thereof when compared to a second plant of the same variety lacking the modification.

2. The plant of claim 1, wherein the modification comprises a deletion, a substitution, or an insertion, or a combination thereof.

3. The plant of claim 1, wherein the modification is to an endogenous gene or a regulatory element thereof.

4. The plant of claim 1, wherein the modification is to the MsFT gene.

5. The plant of claim 1, wherein the modification comprises a recombinant DNA molecule integrated into the genome of the plant, wherein the recombinant DNA molecule comprises all or a portion of a nucleic acid sequence at least 95% identical to SEQ ID NO:1, and wherein the transcription of all or a portion of the recombinant DNA molecule suppresses the expression of the MsFT gene in said plant through an RNA interference pathway.

6. The plant of claim 1, wherein the plant is an agronomically acceptable plant that exhibits increased vigor, biomass, percent dry matter, leaf to stem ratio, crude protein, or percent neutral detergent fiber digestibility, or decreased percent acid detergent fiber, percent acid detergent lignin, or percent neutral detergent fiber, or a combination thereof when compared to the second plant of the same variety lacking the modification.

7. The plant of claim 1, wherein the modification is produced by irradiation, transposon insertion, chemical mutagenesis, genetic transformation, or genome-editing, or a combination thereof.

8. A recombinant DNA molecule comprising a promoter functional in alfalfa operably linked to all or a portion of a polynucleotide molecule at least 95% identical to SEQ ID NO:1 in sense or antisense orientations or both, wherein the transcription of all or a portion of the recombinant DNA molecule in an alfalfa plant results in suppressing the expression of the MsFT gene in said plant.

9. The recombinant DNA molecule of claim 8, wherein said all or a portion of the polynucleotide molecule is present in sense and antisense orientation and said transcription of all or a portion of the recombinant DNA molecule produces a double stranded RNA.

10. The recombinant DNA molecule of claim 8, further defined as comprising a DNA sequence with at least 95% sequence identity to SEQ ID NO:4 or 5, or a fragment thereof.

11. The recombinant DNA molecule of claim 8, wherein the recombinant DNA molecule is operably linked to a heterologous promoter.

12. The recombinant DNA molecule of claim 8, wherein said all or a portion of the polynucleotide molecule of SEQ ID NO:1 corresponds to the segment B region of the MsFT gene.

13. The recombinant DNA molecule of claim 8, wherein the molecule comprises a molecular cloning, transformation, transfection, or transduction vector, or a combination thereof.

14. A transgenic alfalfa plant, plant part, seed, or plant cell comprising the recombinant DNA molecule of claim 8.

15. The plant of claim 14, wherein the plant exhibits improved forage production, quality, digestibility, or a combination thereof when compared to a plant of the same variety lacking the recombinant DNA molecule.

16. The plant of claim 15, wherein the plant exhibits increased vigor, biomass, percent dry matter, leaf to stem ratio, crude protein, or percent neutral detergent fiber digestibility, or decreased percent acid detergent fiber, percent acid detergent lignin, or percent neutral detergent fiber, or a combination thereof when compared to the plant of the same variety lacking the recombinant DNA molecule.

17. A method for producing a transgenic plant, the method comprising the steps of:
(a) transforming a plant cell with the recombinant DNA molecule of claim 8; and
(b) regenerating a transgenic plant from the transformed plant cell.

18. A method for producing a transgenic plant, the method comprising the steps of:
(a) transforming a plurality of plant cells with the recombinant DNA molecule of claim 8;
(b) regenerating a plurality of transgenic plants from the transformed plant cells; and
(c) screening the plurality of transgenic plants to select at least a first plant comprising improved forage production, forage quality, or forage digestibility.

19. A plant produced by the method of claim 18.

20. A method of producing an alfalfa plant exhibiting improved forage production, quality, or digestibility, or a combination thereof, the method comprising crossing the plant of claim 1 with itself or another alfalfa plant.

21. A method of producing an alfalfa plant exhibiting improved forage production, quality, or digestibility, or a combination thereof, the method comprising crossing a plant comprising the recombinant DNA molecule of claim 8 with itself or another alfalfa plant.

22. A method of generating a modified alfalfa plant exhibiting improved forage production, quality, digestibility, or a combination thereof, the method comprising introducing a mutation into the MsFT gene in said plant that results in reduced expression of said gene.

23. The method of claim 22, wherein introducing said mutation comprises genome-editing or genetic transformation.


SEQUENCE LISTING (10 sequences)

SEQ ID NO: 1 - DNA, 1737 bp, Medicago sativa L.
atggccggtagcagtaggaatccactggctgtagggcgtgtaataggggatgtgatagactcctttgaaagttccattcctctccgagtgacctatggtaataaagatgtgaataatggttgtgagctcaaaccttctcaaattggcaatcaacccagagtgagtgttggtggaaacgatctcagaaacctctacaccctagtaatttacattatatagtcttatttctttgttgtttttgaaatacatttttatatttaacacaagtctatagttagcctattatcataatgtagtttctaatttcttgctctatttttataggttatggtggatcctgattcacctagcccaagtaaccccacttttaaggagtacctccactggtgagtaaatcaattacatgccttatgctgcttatgatttacctaagacttttgttaaataatatatagctaacactcttctctcaataaactttaggttggtgactgatattccaggaaccactgaagtcactttcggtatggatattttatacatgaatctgttacttgttaggtcaaataatataacaagaactcaatgggcaactattctttcatttgtgactttttacaaataaagttagagggtatctgaagcatttgagtaatcacattactgagtgtgtttggatgagatacttagaaattttagagaaactagaattttaagaattcaaatacttcaattgaattctcttcatttctaatttttcttgcttggatagaataatatgattctccaatcttaaacttggattatttttatgaaatttcagaaaagttgagggattaacttcaaaatatgaaaaaaggactaatttacaatttttaaaaattatagggaccaatttgcaatttataaaaaactcataggaaccaatttgcagtttttgaaaatttaaagggaccaatttgtatttttttaaaatatagggaccaaatttcaaaattttaataatatatagggactcatgtgtaatttttttttttaaaatataggaaccaatttgcataatttgaaagtgttaaaaagatcaatttgaaatttacattgtatagactattagagtaatgaagaaattttaatttttttggtttttagctgttatttgaaatttcaaaaatttagctttgaattgaagttgagtttaacataatcaccacatgccaatatgattttggtaaaaaccaatgcattagaacttcaaaaaaacattgtgttgccatagttttgtcaaattcagagtcaatccaaacatctaagaaatgaaaaataaatccattgtagcatattcatttattgtttatgtgaataacaaattaagaacaacactagaattattttcataattttacttattaaagagaaagactacatggttatgcctattggtagaattgttcatttgcaaagttacattttccaccctagttaattttgagtttgattgatttgcacataggtaatgaggttgtaaattatgaaaggccacgacccacttcggggatccatcgtttcgtgtttgtcctatttcatcaacaatgtagacaaagggtttatgctccaggatggcgacaaaatttcaacacaagagaatttgctgaactctacaatcttggatcacctgttgctgctgtcttcttcaattgtcaaagggagagtggctctggcggaagaacctttagataa

SEQ ID NO: 2 - DNA, 531 bp, Medicago sativa L.
atggccggtagcagtaggaatccactggctgtagggcgtgtaataggggatgtgatagactcctttgaaagttccattcctctccgagtgacctatggtaataaagatgtgaataatggttgtgagctcaaaccttctcaaattggcaatcaacccagagtgagtgttggtggaaacgatctcagaaacctctacaccctagttatggtggatcctgattcacctagcccaagtaaccccacttttaaggagtacctccactggttggtgactgatattccaggaaccactgaagtcactttcggtaatgaggttgtaaattatgaaaggccacgacccacttcggggatccatcgtttcgtgtttgtcctatttcatcaacaatgtagacaaagggtttatgctccaggatggcgacaaaatttcaacacaagagaatttgctgaactctacaatcttggatcacctgttgctgctgtcttcttcaattgtcaaagggagagtggctctggcggaagaacctttagataa

SEQ ID NO: 3 - DNA, 531 bp, Medicago sativa L.
taccggccatcgtcatccttaggtgaccgacatcccgcacattatcccctacactatctgaggaaactttcaaggtaaggagaggctcactggataccattatttctacacttattaccaacactcgagtttggaagagtttaaccgttagttgggtctcactcacaaccacctttgctagagtctttggagatgtgggatcaataccacctaggactaagtggatcgggttcattggggtgaaaattcctcatggaggtgaccaaccactgactataaggtccttggtgacttcagtgaaagccattactccaacatttaatactttccggtgctgggtgaagcccctaggtagcaaagcacaaacaggataaagtagttgttacatctgtttcccaaatacgaggtcctaccgctgttttaaagttgtgttctcttaaacgacttgagatgttagaacctagtggacaacgacgacagaagaagttaacagtttccctctcaccgagaccgccttcttggaaatctatt

SEQ ID NO: 4 - DNA, 248 bp, Medicago sativa L.
acctccactggttggtgactgatattccaggaaccactgaagtcactttcggtaatgaggttgtaaattatgaaaggccacgacccacttcggggatccatcgtttcgtgtttgtcctatttcatcaacaatgtagacaaagggtttatgctccaggatggcgacaaaatttcaacacaagagaatttgctgaactctacaatcttggatcacctgttgctgctgtcttcttcaattgtcaaagggag

SEQ ID NO: 5 - DNA, 248 bp, Medicago sativa L.
tggaggtgaccaaccactgactataaggtccttggtgacttcagtgaaagccattactccaacatttaatactttccggtgctgggtgaagcccctaggtagcaaagcacaaacaggataaagtagttgttacatctgtttcccaaatacgaggtcctaccgctgttttaaagttgtgttctcttaaacgacttgagatgttagaacctagtggacaacgacgacagaagaagttaacagtttccctc

SEQ ID NO: 6 - RNA, 887 nt, Medicago sativa L.
ucaccauuuuguguauagaagugugagaacugagaaccacauauggccgguagcaguaggaauccacuggcuguagggcguguaauaggggaugugauagacuccuuugaaaguuccauuccucuccgagugaccuaugguaauaaagaugugaauaaugguugugagcucaaaccuucucaaauuggcaaucaacccagagugaguguugguggaaacgaucucagaaaccucuacacccuaguuaugguggauccugauucaccuagcccaaguaaccccacuuuuaaggaguaccuccacugguuggugacugauauuccaggaaccacugaagucacuuucgguaaugagguuguaaauuaugaaaggccacgacccacuucggggauccaucguuucguguuuguccuauuucaucaacaauguagacaaaggguuuaugcuccaggauggcgacaaaauuucaacacaagagaauuugcugaacucuacaaucuuggaucaccuguugcugcugucuucuucaauugucaaagggagaguggcucuggcggaagaaccuuuagauaauuauuauauuaacuauaugaaauuaaaauuaaaguggaagcuuagggcugguuugguaucacaauguaucaccaugagucaagacauuguaauaucaaacauaugcguaguuaaguuaguuaguaguuaaauacugcaacacccaaauaaaaguuaauuauauuuacauauacacauuauacacucacacacgccaaauuaauacacaauuguaaaaguagcaaauacuaaguauagauaugugugucguuuuauguuuguguauguauaauguaucguauauuaucaucaaucaauaaagagcuucccacugc

SEQ ID NO: 7 - protein, 176 aa, Medicago sativa L.
MAGSSRNPLAVGRVIGDVIDSFESSIPLRVTYGNKDVNNGCELKPSQIGNQPRVSVGGNDLRNLYTLVMVDPDSPSPSNPTFKEYLHWLVTDIPGTTEVTFGNEVVNYERPRPTSGIHRFVFVLFHQQCRQRVYAPGWRQNFNTREFAELYNLGSPVAAVFFNCQRESGSGGRTFR

SEQ ID NO: 8 - DNA, 25 bp, Artificial sequence
  Primer
caccacctccactggttggtgactg

SEQ ID NO: 9 - DNA, 24 bp, Artificial sequence
  Primer
ctccctttgacaattgaagaagac

SEQ ID NO: 10 - DNA, 531 bp, Medicago truncatula
atggctggtagcagtaggaatccactagctgtagggcgtgtaataggggatgtaatagactcatttgaaaattccattcctcttcgagtgacctatggtaatagggatgtgaataatggttgtgagctcaaaccttctcaaattggaaatcaaccgagagtgagtgttggtggaaacgatctcagaaacctctacaccctagttatggtggatcctgattcacctagcccaagtaaccccacttttaaggagtacctccactggttggtgactgatattccaggaaccactgaagtcactttcggcaatgaggttgtaaattatgaaaggccacgacccacttcagggatccatcgtttcgtgtttgtcttattccgtcaacagtgtagacaaagggtttatgctccaggatggcgacaaaatttcaacacaagagaatttgctgaactctacaatcttggatcacctgttgctgctgtcttcttcaattgtcagagggagagtggctctggaggaagaacctttagataa